(19) United States
(12) Patent Application Publication
(10) Pub. No.: US 2024/0079100 A1
(43) Pub. Date: Mar. 7, 2024

(54) MEDICAL SUPPORT DEVICE, MEDICAL SUPPORT METHOD, AND PROGRAM

(71) Applicant: FUJIFILM CORPORATION, Tokyo (JP)

(72) Inventor: Takeharu NAKAZATO, Kanagawa (JP)

(21) Appl. No.: 18/307,831

(22) Filed: Apr. 27, 2023

(30) Foreign Application Priority Data
May 9, 2022 (JP) 2022-077138

(51) Int. Cl. G16H 10/40; G16H 15/00

(52) U.S. Cl. CPC G16H 10/40; G16H 15/00

(57) ABSTRACT

A medical support device includes a processor. In a case in which a tissue is collected at a plurality of first positions in an organ, the processor outputs collection-related information which is information related to a collection state of the tissue at each of the first positions.

### CROSS-REFERENCE TO RELATED APPLICATION

This application claims priority from Japanese Patent Application No. 2022-077138, filed May 9, 2022, the disclosure of which is incorporated herein by reference in its entirety.

### BACKGROUND

### Technical Field

The technology of the present disclosure relates to a medical support device, a medical support method, and a non-transitory storage medium storing a program.

### Related Art

WO2017/175494A discloses a diagnostic imaging system comprising an acquisition unit that acquires diagnostic images of a subject and an association unit that associates a diagnostic image, in which a collection position in a case in which a specimen is collected from the subject can be identified, among the diagnostic images acquired by the acquisition unit with information for specifying the specimen collected from the subject.

JP2005-007145A discloses a medical image recording device that records endoscope images captured by an endoscope and that comprises a generation unit which generates image data for performing display based on an operator's instruction on a monitor and a combination unit that combines the endoscope images captured by the endoscope with the image data and outputs a combination result to the monitor.

WO2019/088178A discloses a biopsy support device that supports a test using a biological tissue collected by a collection tool which is inserted into an endoscope having an imaging element and is used to collect the biological tissue. The biopsy support device comprises a collection recognition unit that recognizes the collection of the biological tissue by the collection tool on the basis of captured image data captured by the imaging element and an identification information generation unit that generates identification information corresponding to the biological tissue in a case in which the collection recognition unit recognizes the collection of the biological tissue.

### SUMMARY

An embodiment according to the technology of the present disclosure provides a medical support device, a medical support method, and a non-transitory storage medium storing a program that enable a user to ascertain a collection state of a tissue.

According to a first aspect of the technology of the present disclosure, there is provided a medical support device comprising a processor. In a case in which a tissue is collected at a plurality of first positions in an organ, the processor outputs collection-related information which is information related to a collection state of the tissue at each of the first positions.

According to a second aspect of the technology of the present disclosure, in the medical support device according to the first aspect, the collection-related information may include collection completion information which is information indicating completion of the collection of the tissue at each of the first positions.

According to a third aspect of the technology of the present disclosure, in the medical support device according to the first aspect or the second aspect, the collection-related information may include collection number information which is information indicating the number of times the tissue is collected at each of the first positions.

According to a fourth aspect of the technology of the present disclosure, in the medical support device according to the first aspect, the collection-related information may include collection completion information, which is information indicating completion of the collection of the tissue at each of the first positions, and collection number information, which is information indicating the number of times the tissue is collected at each of the first positions.

According to a fifth aspect of the technology of the present disclosure, in the medical support device according to any one of the first to fourth aspects, the collection-related information may include collection identification information which is information for identifying each of a plurality of collections of the tissue in a case in which the collection of the tissue at the first position is performed a plurality of times.

According to a sixth aspect of the technology of the present disclosure, in the medical support device according to the fifth aspect, the collection identification information may be associated with a pathological diagnosis result obtained by the collection of the tissue for each of the plurality of collections of the tissue at the first position.

According to a seventh aspect of the technology of the present disclosure, in the medical support device according to any one of the first to sixth aspects, the collection-related information may be generated on the basis of a medical image showing the collection of the tissue at the first position.

According to an eighth aspect of the technology of the present disclosure, in the medical support device according to the seventh aspect, the medical image may be a real-time image.

According to a ninth aspect of the technology of the present disclosure, in the medical support device according to the seventh aspect or the eighth aspect, the medical image may be an optical image and/or an ultrasound image.

According to a tenth aspect of the technology of the present disclosure, in the medical support device according to any one of the seventh to ninth aspects, the collection-related information may be generated in a case in which the collection of the tissue at the first position is performed in the medical image.

According to an eleventh aspect of the technology of the present disclosure, in the medical support device according to any one of the seventh to tenth aspects, the collection-related information may be associated with the medical image obtained for each of the plurality of first positions.

According to a twelfth aspect of the technology of the present disclosure, in the medical support device according to any one of the first to sixth aspects, the collection-related information may be generated on the basis of an instruction received by a receiving device.

According to a thirteenth aspect of the technology of the present disclosure, in the medical support device according to any one of the first to twelfth aspects, the instruction received by the receiving device may be an instruction with respect to a result generated on the basis of a medical image showing the collection of the tissue at the first position.

According to a fourteenth aspect of the technology of the present disclosure, in the medical support device according to any one of the first to twelfth aspects, the instruction received by the receiving device may be an instruction received in a case in which the collection of the tissue is detected on the basis of a medical image showing the collection of the tissue at the first position.

According to a fifteenth aspect of the technology of the present disclosure, in the medical support device according to any one of the first to fourteenth aspects, the collection state of the tissue indicated by the collection-related information corresponding to the first position may be displayed at each second position which is a position corresponding to the first position in an organ image which is an image showing the organ.

According to a sixteenth aspect of the technology of the present disclosure, in the medical support device according to the fifteenth aspect, the number of times the tissue is collected which is indicated by the collection-related information may be displayed at the second position on a display device.

According to a seventeenth aspect of the technology of the present disclosure, in the medical support device according to any one of the first to sixteenth aspects, an order in which the tissue is collected at the plurality of first positions may be predetermined.

According to an eighteenth aspect of the technology of the present disclosure, there is provided a medical support method comprising: outputting, in a case in which a tissue is collected at a plurality of first positions in an organ, collection-related information which is information related to a collection state of the tissue at each of the first positions.

According to a nineteenth aspect of the technology of the present disclosure, the medical support method according to the eighteenth aspect may further comprise: determining whether or not the tissue has been collected on the basis of a medical image showing the collection of the tissue; receiving a user's instruction with respect to a determination result; and outputting collection number information indicating the number of times the tissue is collected at each of the first positions according to the instruction.

According to a twentieth aspect of the technology of the present disclosure, there is provided a non-transitory storage medium storing a program that causes a computer to execute a process comprising outputting, in a case in which a tissue is collected at a plurality of first positions in an organ, collection-related information which is information related to a collection state of the tissue at each of the first positions.

### BRIEF DESCRIPTION OF THE DRAWINGS

FIG. 1 is a conceptual diagram illustrating an example of an aspect in which an endo scope system is used.

FIG. 2 is a conceptual diagram illustrating an example of an overall configuration of the endoscope system.

FIG. 3 is a conceptual diagram illustrating an example of an aspect in which an insertion portion of a bronchoscope is inserted into a body of a subject.

FIG. 4 is a block diagram illustrating an example of a hardware configuration of an endoscope apparatus.

FIG. 5 is a block diagram illustrating an example of functions of main units of a processor.

FIG. 6 is a conceptual diagram illustrating an example of the content of a biopsy route generation process performed by a biopsy route generation device and the content of a process of a first display control unit.

FIG. 7 is a conceptual diagram illustrating an example of the content of processes of a second display control unit and a third display control unit.

FIG. 8 is a conceptual diagram illustrating an example of the content of processes of a third display control unit, a voice recognition unit, and a collection-related information generation unit.

FIG. 9 is a conceptual diagram illustrating an example of the content of the processes of the third display control unit, the voice recognition unit, and the collection-related information generation unit.

FIG. 10 is a flowchart illustrating an example of a flow of a medical support process.

FIG. 11 is a conceptual diagram illustrating an example of the content of the processes of the third display control unit, the voice recognition unit, and the collection-related information generation unit.

FIG. 12 is a conceptual diagram illustrating an example of the content of the processes of the third display control unit, the voice recognition unit, and the collection-related information generation unit.

FIG. 13 is a conceptual diagram illustrating an example of the content of a process of the collection-related information generation unit.

FIG. 14 is a conceptual diagram illustrating an example of the content of the processes of the third display control unit, an image recognition unit, and the collection-related information generation unit.

FIG. 15 is a conceptual diagram illustrating an example of the content of the processes of the third display control unit, the image recognition unit, and the collection-related information generation unit.

FIG. 16 is a conceptual diagram illustrating an example of the content of the processes of the third display control unit and the voice recognition unit.

FIG. 17 is a conceptual diagram illustrating an example of the content of the processes of the third display control unit and the voice recognition unit.

FIG. 18 is a conceptual diagram illustrating an example of the content of the processes of the third display control unit, the voice recognition unit, and the collection-related information generation unit.

FIG. 19 is a conceptual diagram illustrating an example of the content of the processes of the third display control unit, the image recognition unit, and the collection-related information generation unit.

### DETAILED DESCRIPTION

Hereinafter, examples of embodiments of a medical support device, a medical support method, and a program according to the technology of the present disclosure will be described with reference to the accompanying drawings.

First, terms used in the following description will be described.

CPU is an abbreviation of “central processing unit”. GPU is an abbreviation of “graphics processing unit”. RAM is an abbreviation of “random access memory”. NVM is an abbreviation of “non-volatile memory”. EEPROM is an abbreviation of “electrically erasable programmable read-only memory”. ASIC is an abbreviation of “application specific integrated circuit”. PLD is an abbreviation of “programmable logic device”. FPGA is an abbreviation of “field-programmable gate array”. SoC is an abbreviation of “system-on-a-chip”. SSD is an abbreviation of “solid state drive”. USB is an abbreviation of “universal serial bus”. HDD is an abbreviation of “hard disk drive”. EL is an abbreviation of “electro-luminescence”. I/F is an abbreviation of “interface”. CMOS is an abbreviation of “complementary metal oxide semiconductor”. CCD is an abbreviation of “charge coupled device”. CT is an abbreviation of “computed tomography”. MRI is an abbreviation of “magnetic resonance imaging”. AI is an abbreviation of “artificial intelligence”.

### First Embodiment

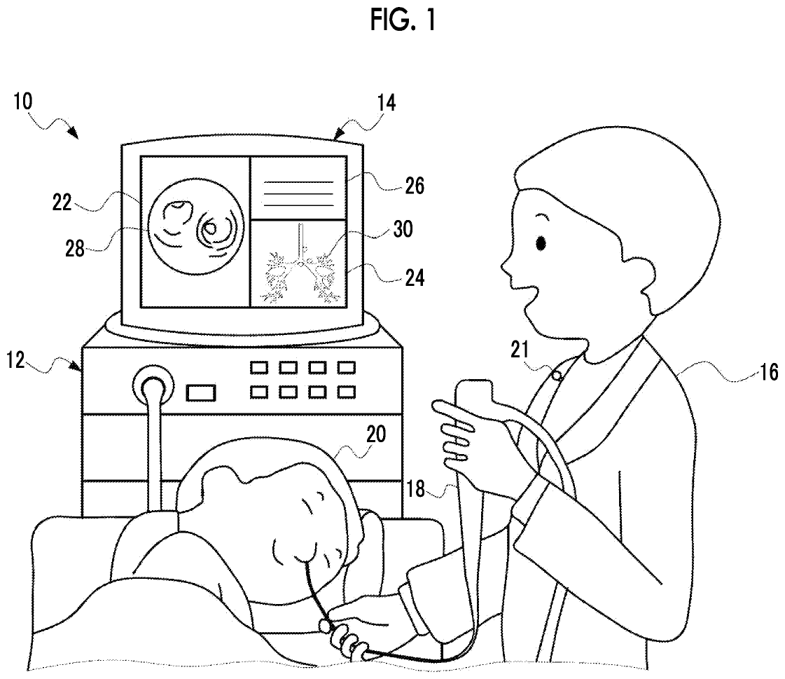

For example, as illustrated in FIG. 1, an endoscope system 10 comprises an endoscope apparatus 12 and a display device 14. The endoscope apparatus 12 is used by a medical worker (hereinafter, referred to as a “user”) such as a doctor 16. The endoscope apparatus 12 is an apparatus that comprises a bronchoscope 18 (endoscope) and is used to perform a medical treatment for a bronchus of a subject 20 (for example, a patient) through the bronchoscope 18.

The bronchoscope 18 is inserted into the bronchus of the subject 20 by the doctor 16, images the inside of the bronchus, acquires an image showing an aspect of the inside of the bronchus, and outputs the image. In the example illustrated in FIG. 1, an aspect in which the bronchoscope 18 is inserted into a body through a nostril of the subject 20 is illustrated. In the example illustrated in FIG. 1, although the bronchoscope 18 is inserted into the body through the nostril of the subject 20, this is only an example, and the bronchoscope 18 may be inserted into the body through a mouth of the subject 20.

The endoscope apparatus 12 comprises a microphone 21. The microphone 21 acquires a voice uttered by the doctor 16 and outputs a voice signal indicating the acquired voice to a predetermined output destination. An example of the microphone 21 is a pin microphone. In the example illustrated in FIG. 1, the microphone 21 is attached to a collar of the doctor 16. Further, the microphone 21 may be disposed at any position as long as it can acquire the voice of the doctor 16. A microphone having directivity toward the mouth of the doctor 16 is preferable. In the example illustrated in FIG. 1, the pin microphone is given as an example of the microphone 21. However, this is only an example, and the microphone 21 may be other types of microphones such as a stand microphone and a bone conduction microphone.

The display device 14 displays various types of information including images. Examples of the display device 14 include a liquid crystal display and an EL display. A plurality of screens are displayed side by side on the display device 14. In the example illustrated in FIG. 1, a first screen 22, a second screen 24, and a third screen 26 are illustrated as an example of a plurality of screens.

An endoscope image 28 obtained by imaging the bronchus of the subject 20 with the bronchoscope 18 is displayed on the first screen 22. An example of the endoscope image 28 is a video image (for example, a live view image). An organ image 30 is displayed on the second screen 24. An example of the organ image 30 is a still image showing the entire bronchi. The organ image 30 is a virtual image showing the entire virtual bronchi that imitate the bronchi observed by the doctor 16 through the endoscope image 28. For example, information related to the subject 20 and/or information related to the operation of the endoscope apparatus 12 is displayed on the third screen 26.

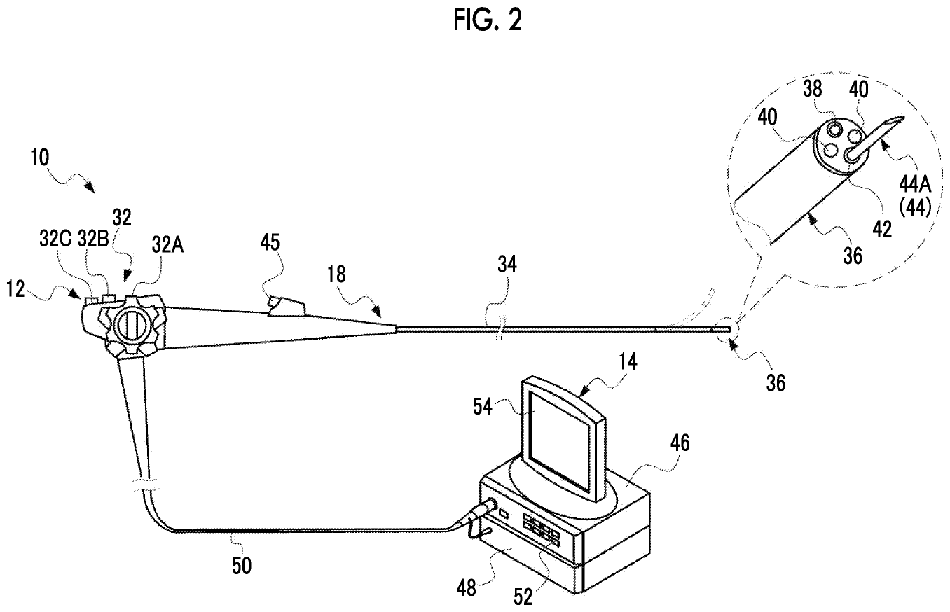

For example, as illustrated in FIG. 2, the bronchoscope 18 comprises an operation unit 32 and an insertion portion 34. The operation unit 32 comprises a rotation operation knob 32A, an air and water supply button 32B, and a suction button 32C. The insertion portion 34 is formed in a tubular shape. The outer contour of the insertion portion 34 in a cross-sectional view has a circular shape. The rotation operation knob 32A of the operation unit 32 is operated to partially bend the insertion portion 34 or to rotate the insertion portion 34 about an axis of the insertion portion 34. As a result, the insertion portion 34 is moved to a back side of the body while being bent according to the shape of the inside of the body (for example, the shape of the bronchus) or while being rotated about the axis of the insertion portion 34 according to an internal part of the body. In addition, the air and water supply button 32B is operated to supply water or air into the body from a distal end part 36, and the suction button 32C is operated to draw water or air in the body.

The distal end part 36 of the insertion portion 34 is provided with a camera 38, an illumination device 40, and a treatment tool opening 42. The camera 38 images the inside of the bronchus. An example of the camera 38 is a CMOS camera. However, this is only an example, and the camera 38 may be other types of cameras such as CCD cameras. The illumination device 40 irradiates the inside of the bronchus with light (for example, visible light). The treatment tool opening 42 is an opening through which a treatment tool 44 protrudes from the distal end part 36. The treatment tool 44 is inserted into the insertion portion 34 through a treatment tool insertion opening 45. The treatment tool 44 passes through the insertion portion 34 and protrudes into the bronchus from the treatment tool opening 42. In the example illustrated in FIG. 2, as the treatment tool 44, a puncture needle 44A protrudes from the treatment tool opening 42. Although, here, the puncture needle 44A is given as an example of the treatment tool 44, this is only an example, and the treatment tool 44 may be, for example, grasping forceps and/or a knife.

The endoscope apparatus 12 comprises a control device 46 and a light source device 48. The bronchoscope 18 is connected to the control device 46 and the light source device 48 through a cable 50. The control device 46 is a device that controls the entire endoscope apparatus 12. The light source device 48 is a device that emits light under the control of the control device 46 and that supplies the light to the illumination device 40.

The control device 46 is provided with a plurality of hard keys 52. The plurality of hard keys 52 receive an instruction from the user. A touch panel 54 is provided on the screen of the display device 14. The touch panel 54 is electrically connected to the control device 46 and receives an instruction from the user. The display device 14 is also electrically connected to the control device 46.

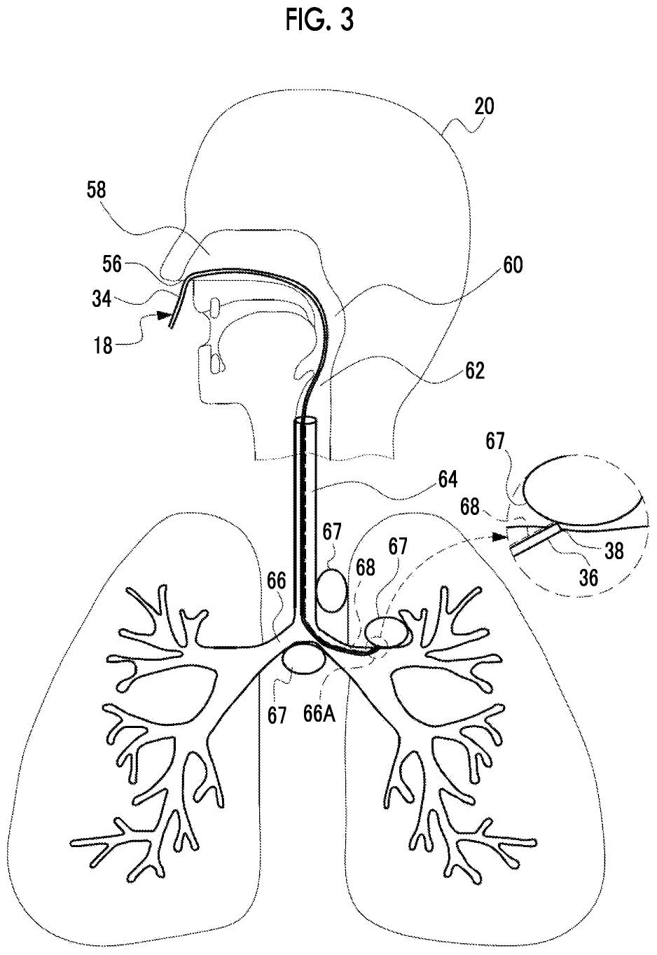

For example, as illustrated in FIG. 3, the insertion portion 34 of the bronchoscope 18 is inserted into a bronchus 66 from a nostril 56 of the subject 20 through a nasal cavity 58, a pharynx 60, a larynx 62, and a trachea 64. The distal end part 36 is moved to a back side of the bronchus 66 along a scheduled route 68 in the bronchus 66. The distal end part 36 moved to the back side of the bronchus 66 eventually reaches a target position 66A in the bronchus 66 (for example, an interior wall corresponding to a lymph node 67 in an edge part of the bronchus 66). The bronchus 66 and the lymph node 67 are examples of “an organ” according to the technology of the present disclosure. In a case in which the distal end part 36 reaches the target position 66A, a treatment (for example, the collection of a sample) is performed by the treatment tool 44 of the distal end part 36. While the distal end part 36 is inserted into the body of the subject 20, the camera 38 images the inside of the bronchus 66 at a predetermined frame rate. An example of the predetermined frame rate is several tens of frames/second (for example, 30 frames/second or 60 frames/second).

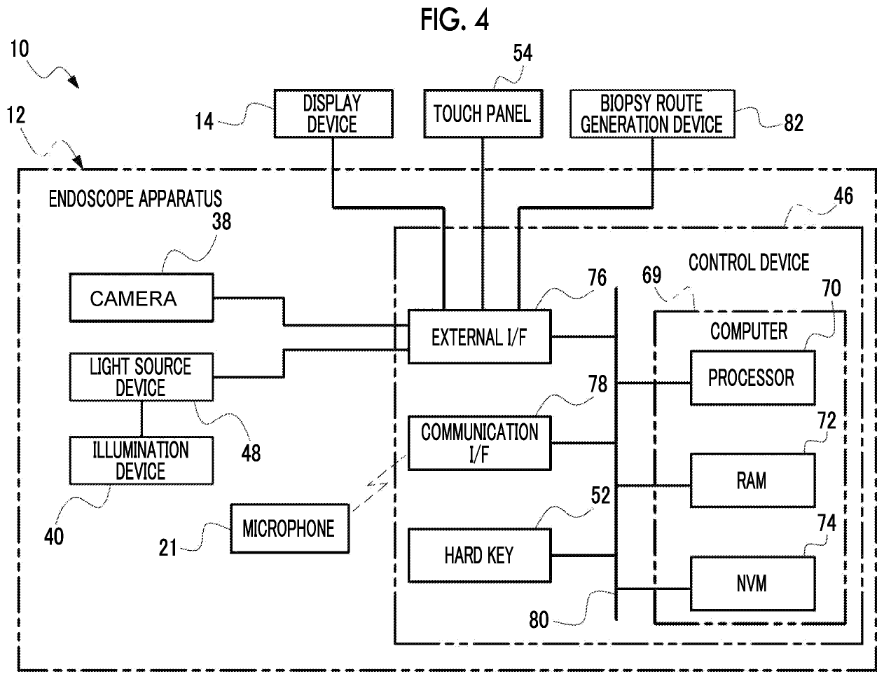

For example, as illustrated in FIG. 4, the control device 46 comprises a computer 69. The computer 69 is an example of a “medical support device” and a “computer” according to the technology of the present disclosure. The computer 69 comprises a processor 70, a RAM 72, and an NVM 74. The processor 70, the RAM 72, and the NVM 74 are electrically connected to each other. The processor 70 is an example of a “processor” according to the technology of the present disclosure.

The control device 46 comprises the hard keys 52, an external I/F 76, and a communication I/F 78. The hard keys 52, the processor 70, the RAM 72, the NVM 74, the external I/F 76, and the communication OF 78 are connected to a bus 80.

For example, the processor 70 includes a CPU and a GPU and controls the entire control device 46. The GPU operates under the control of the CPU and performs various graphic-based processes. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 is a flash memory (for example, an EEPROM and/or an SSD). In addition, the flash memory is only an example and may be other non-volatile storage devices, such as HDDs, or a combination of two or more types of non-volatile storage devices.

The hard keys 52 receive an instruction from the user and output a signal indicating the received instruction to the processor 70. Therefore, the instruction received by the hard keys 52 is recognized by the processor 70.

The external I/F 76 transmits and receives various types of information between a device (hereinafter, also referred to as an “external device”) outside the control device 46 and the processor 70. An example of the external I/F 76 is a USB interface.

As one of the external devices, the camera 38 is connected to the external I/F 76, and the external I/F 76 transmits and receives various types of information between the camera 38 and the processor 70. The processor 70 controls the camera 38 through the external I/F 76. In addition, the processor 70 acquires the endoscope image 28 (see FIG. 1) obtained by imaging the inside of the bronchus 66 with the camera 38 through the external I/F 76.

As one of the external devices, the light source device 48 is connected to the external I/F 76, and the external I/F 76 transmits and receives various types of information between the light source device 48 and the processor 70. The light source device 48 supplies light to the illumination device 40 under the control of the processor 70. The illumination device 40 performs irradiation with the light supplied from the light source device 48.

As one of the external devices, the display device 14 is connected to the external I/F 76, and the processor 70 controls the display device 14 through the external I/F 76 such that the display device 14 displays various types of information.

As one of the external devices, the touch panel 54 is connected to the external I/F 76, and the processor 70 acquires the instruction received by the touch panel 54 through the external I/F 76.

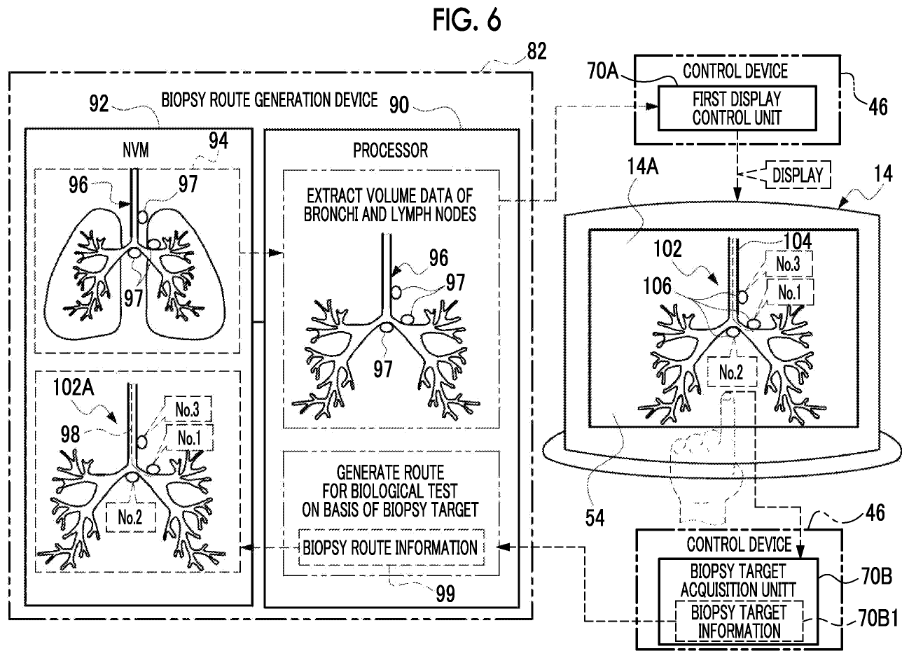

As one of the external devices, a biopsy route generation device 82 is connected to the external I/F 76. An example of the biopsy route generation device 82 is a server. The server is only an example, and the biopsy route generation device 82 may be a personal computer. The biopsy route generation device 82 calculates a bronchial pathway 98 (see FIG. 6) used for a biological test or generates the organ image 30 (see FIG. 1). The external I/F 76 transmits and receives various types of information between the biopsy route generation device 82 and the processor 70. The processor 70 requests the biopsy route generation device 82 to provide a service (for example, to generate and provide the organ image 30) through the external I/F 76 or acquires the organ image 30 from the biopsy route generation device 82 through the external I/F 76.

The communication I/F 78 is an interface including, for example, an antenna and a communication processor. For example, the communication I/F 78 performs wireless communication with a communication device using a communication system, such as Wi-Fi (registered trademark) or Bluetooth (registered trademark), to transmit and receive various types of information between the communication device and the processor 70. An example of the communication device is the microphone 21. The processor 70 acquires a voice signal from the microphone 21 through the communication OF 78.

However, in endoscopy, a biological test (hereinafter, also simply referred to as a “biopsy”) that directly collects tissues in the body may be performed. The biological test may be sequentially performed on a plurality of parts in the body during one medical treatment. In this case, the medical worker needs to ascertain the progress of tissue collection or to check whether or not there is any omission in collection while performing a collection operation in each part.

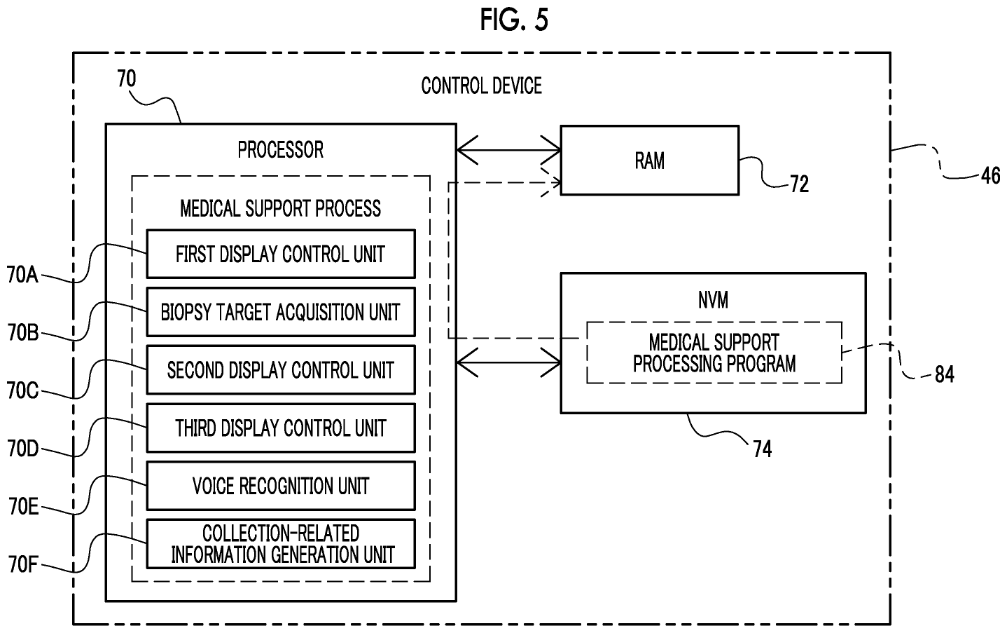

Therefore, in view of these circumstances, in this embodiment, for example, as illustrated in FIG. 5, the processor 70 performs a medical support process. A medical support processing program 84 is stored in the NVM 74. The medical support processing program 84 is an example of a “program” according to the technology of the present disclosure.

The processor 70 reads the medical support processing program 84 from the NVM 74 and executes the read medical support processing program 84 on the RAM 72 to perform the medical support process. The processor 70 operates as a first display control unit 70A, a biopsy target acquisition unit 70B, a second display control unit 70C, a third display control unit 70D, a voice recognition unit 70E, and a collection-related information generation unit 70F according to the medical support processing program 84 to implement the medical support process.

For example, as illustrated in FIG. 6, the biopsy route generation device 82 comprises a processor 90, an NVM 92, and a RAM (not illustrated). The processor 90 executes a biopsy route generation processing program (not illustrated) on the RAM to perform a biopsy route generation process. Hereinafter, an example of the content of the biopsy route generation process performed by the processor 90 and the content of the biopsy route generation process performed by the processor 70 of the control device 46 will be described with reference to FIG. 6.

In the biopsy route generation device 82, volume data 94 is stored in the NVM 92. The volume data 94 is a three-dimensional image that is defined by voxels in a stack of a plurality of two-dimensional slice images obtained by imaging the whole body or a part (for example, a chest) of the body of the subject 20 with a modality. The position of each voxel is specified by three-dimensional coordinates. An example of the modality is a CT apparatus. The CT apparatus is only an example, and other examples of the modality are an MRI apparatus and an ultrasound diagnostic apparatus.

The volume data 94 includes bronchial volume data 96 which is a three-dimensional image showing the trachea 64 and the bronchi 66 of the subject 20. In addition, the volume data 94 includes lymph node volume data 97 which is a three-dimensional image showing the lymph node 67 of the subject 20.

The processor 90 extracts the bronchial volume data 96 and the lymph node volume data 97 from the volume data 94. In the control device 46, the first display control unit 70A acquires the bronchial volume data 96 and the lymph node volume data 97 from the NVM 92 of the biopsy route generation device 82 through the processor 90. Then, the first display control unit 70A displays a selection image 102 on the display device 14. The selection image 102 is generated by the first display control unit 70A on the basis of the bronchial volume data 96 and the lymph node volume data 97. The selection image 102 is a rendered image of the bronchial volume data 96 and the lymph node volume data 97 on a screen 14A of the display device 14. The selection image 102 is a rendered image obtained by integrating a bronchial image 104 and a lymph node image 106. The bronchial image 104 is a rendered image corresponding to the bronchial volume data 96, and the lymph node image 106 is a rendered image corresponding to the lymph node volume data 97.

In a state in which the selection image 102 is displayed on the screen 14A of the display device 14, the touch panel 54 receives a biopsy target selection instruction from the user. In the example illustrated in FIG. 6, an aspect in which the biopsy target selection instruction is input to the touch panel 54 by a finger of the user is illustrated. The biopsy target selection instruction is an instruction to select a lymph node image 106 showing the lymph node 67 to be subjected to the biological test from a plurality of lymph node images 106. In addition, the biopsy target selection instruction may include an instruction to select an order in which the biological test is performed. FIG. 6 illustrates an example in which a plurality of lymph node images 106 are selected in the order of No. 1, No. 2, and No. 3. In addition, here, the example in which the biopsy target selection instruction is received through the touch panel 54 has been described. However, this is only an example. For example, the biopsy target selection instruction may be received through a mouse and/or a keyboard.

In addition, here, the example in which the lymph node 67 to be subjected to the biological test is selected by the user has been described. However, this is only an example. For example, the lymph node 67 to be subjected to the biological test may be predetermined, or the processor 90 may select the lymph node 67 to be subjected to the biological test on the basis of the lymph node volume data 97.

In the control device 46, the biopsy target acquisition unit 70B generates biopsy target information 70B1 on the basis of the selection result of the lymph node image 106 through the touch panel 54. The biopsy target information 70B1 is information in which three-dimensional coordinates before rendering (that is, three-dimensional coordinates in the bronchial volume data 96 and the lymph node volume data 97) and two-dimensional coordinates after rendering (that is, two-dimensional coordinates in the selection image 102) are associated with each other. In addition, the biopsy target information 70B1 includes information related to the order in which the lymph node images 106 are selected through the touch panel 54.

In the biopsy route generation device 82, the processor 90 acquires the biopsy target information 70B1 from the biopsy target acquisition unit 70B of the control device 46. In addition, the processor 90 performs a thinning process on the bronchial volume data 96 to generate a plurality of bronchial pathways 98. The bronchial pathway 98 is a three-dimensional line that passes through the center of a virtual bronchus (hereinafter, also referred to as a “virtual bronchus”) indicated by the bronchial volume data 96 in a cross-sectional view. The three-dimensional line passing through the center of the virtual bronchus in a cross-sectional view is obtained by thinning the bronchial volume data 96.

The processor 90 generates biopsy route information 99 on the basis of the biopsy target information 70B1 and the bronchial pathway 98. The biopsy route information 99 is information indicating the route 68 in a case in which the biological test is performed. For example, the processor 90 generates the biopsy route information 99 by selecting the shortest bronchial pathway for the biological test from the plurality of bronchial pathways 98 on the basis of the three-dimensional coordinates in the lymph node volume data 97 indicated by the biopsy target information 70B1. In addition, the biopsy route information 99 includes an order in which the lymph nodes 67 are collected in the process of performing the biological test. Further, the example in which the processor 90 generates the biopsy route information 99 on the basis of the biopsy target information 70B1 and the bronchial pathway 98 has been described here. However, this is only an example. For example, the processor 90 may generate the biopsy route information 99 according to the route of the biological test input to a receiving device by the user.

In addition, the processor 90 generates a confirmation image 102A which is an image showing the bronchial pathway 98 indicated by the biopsy route information 99 and the order in which tissues are collected from the lymph nodes 67. The confirmation image 102A is, for example, a two-dimensional image obtained by displaying the bronchial pathway 98 and the order of collection on the selection image 102. In addition, the example in which the confirmation image 102A is generated using the selection image 102 has been described here. However, this is only an example. The confirmation image 102A is an image different from the selection image 102 and may be a two-dimensional image showing an organ or a three-dimensional model image. The processor 90 stores the generated confirmation image 102A in the NVM 92. The confirmation image 102A is an example of an “organ image” according to the technology of the present disclosure.

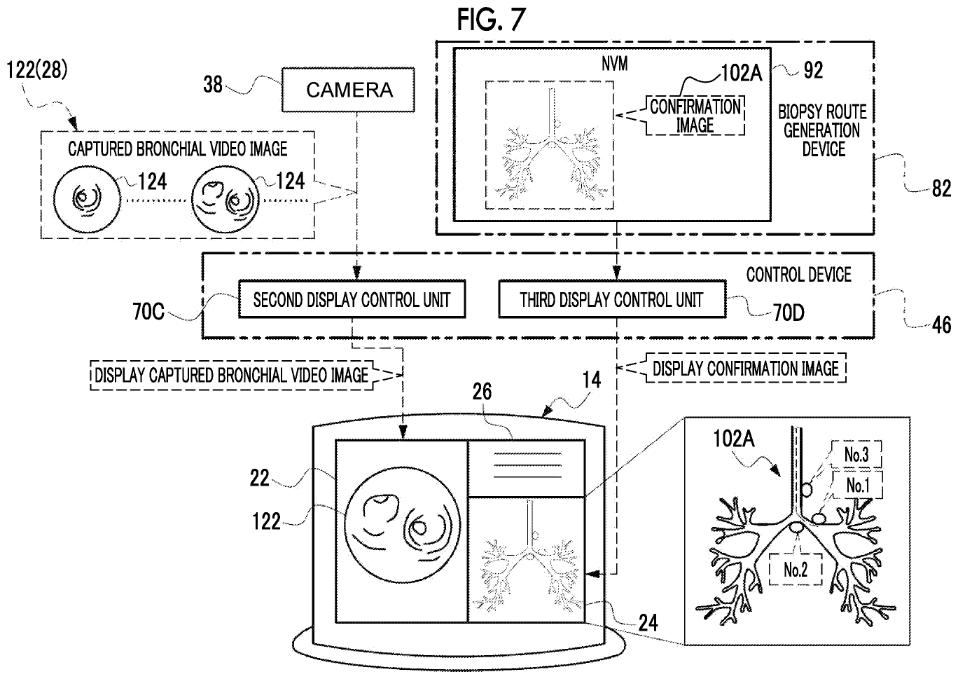

For example, as illustrated in FIG. 7, in the control device 46, the second display control unit 70C acquires a captured bronchial video image 122 from the camera 38. The captured bronchial video image 122 is an example of the endoscope image 28 illustrated in FIG. 1. The captured bronchial video image 122 is a video image (here, for example, a live view image) obtained by imaging the inside of the trachea 64 and the inside of the bronchus 66 (see FIG. 3) along the route 68 (see FIG. 3) with the camera 38. The captured bronchial video image 122 includes a plurality of frames 124 obtained by performing imaging according to a predetermined frame rate from a starting point to an end point of the route 68. The second display control unit 70C outputs the plurality of frames 124 to the display device 14 in time series to display the captured bronchial video image 122 on the first screen 22 of the display device 14.

In the control device 46, the third display control unit 70D acquires the confirmation image 102A from the NVM 92 of the biopsy route generation device 82. Then, the third display control unit 70D outputs the confirmation image 102A to the display device 14 to display the confirmation image 102A on the second screen 24 of the display device 14. In addition, the reception of a start instruction (that is, an instruction to start the display of the confirmation image 102A) from the user by the receiving device, such as the microphone 21, the touch panel 54, or the hard keys 52, (hereinafter, also simply referred to as a “receiving device”) is given as an example of a trigger for causing the display device 14 to start the display of the confirmation image 102A (that is, a trigger for the third display control unit 70D to start the output of the confirmation image 102A).

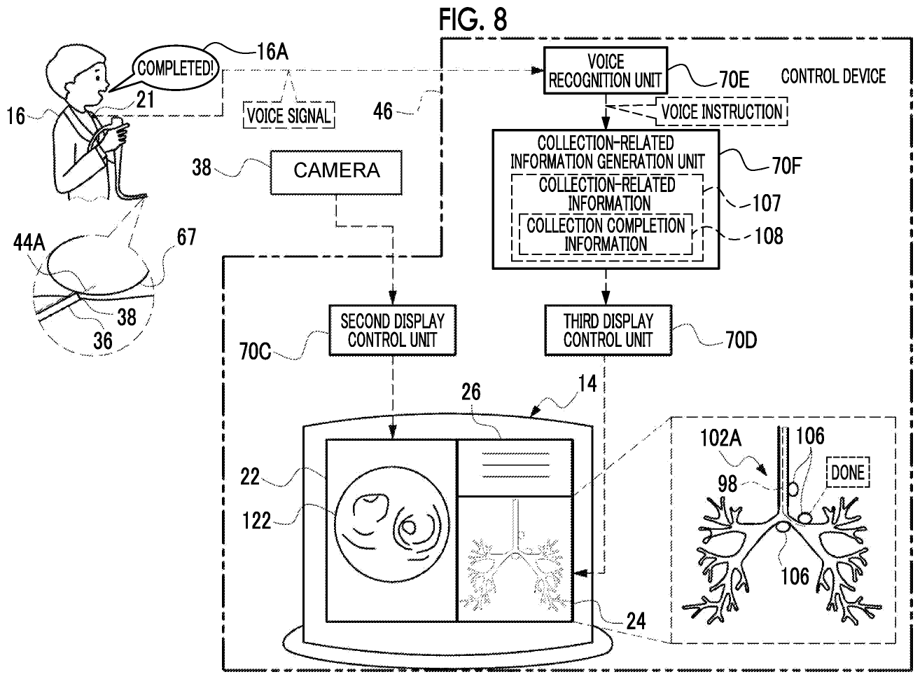

For example, as illustrated in FIG. 8, the microphone 21 outputs a voice 16A uttered by the doctor 16 as a voice signal to the voice recognition unit 70E. The voice 16A uttered by the doctor 16 is an example of an “instruction” according to the technology of the present disclosure, and the microphone 21 is an example of the “receiving device” according to the technology of the present disclosure. The voice recognition unit 70E recognizes the voice indicated by the voice signal input from the microphone 21. The voice is recognized using a known technique. In a case in which the voice recognition unit 70E recognizes a voice instruction which is an instruction by the voice of the doctor 16, the collection-related information generation unit 70F receives the voice instruction output by the voice recognition unit 70E. The voice instruction includes an instruction indicating the completion of the biological test. For example, a voice instruction “completed” issued by the doctor 16 is an instruction indicating that the collection of the tissues of the lymph node 67 by the puncture needle 44A has been completed. In addition, here, for convenience of explanation, the voice instruction “completed” is given as an example. However, this is only an example, and any voice instruction may be used as long as it can specify that the biological test has been completed.

The collection-related information generation unit 70F generates collection-related information 107 according to the voice instruction. The collection-related information 107 is information related to the collection state of the tissues for each of the plurality of lymph nodes 67. The collection-related information 107 is an example of “collection-related information” according to the technology of the present disclosure. FIG. 8 illustrates an example in which the collection-related information generation unit 70F generates collection completion information 108 as the collection-related information 107. The collection completion information 108 is information indicating that the collection of the tissues has been completed for one lymph node 67 among the plurality of lymph nodes 67 to be collected. The collection completion information 108 is an example of “collection completion information” according to the technology of the present disclosure. The collection-related information generation unit 70F outputs the collection completion information 108 to the third display control unit 70D.

In the example illustrated in FIG. 8, an aspect in which the third display control unit 70D displays the confirmation image 102A on the second screen 24 is illustrated. The third display control unit 70D acquires the collection completion information 108 from the collection-related information generation unit 70F. The third display control unit 70D updates the confirmation image 102A displayed on the second screen 24 according to the collection completion information 108. The third display control unit 70D adds display indicating that the collection of the tissues has been completed to the lymph node image 106 corresponding to the lymph node 67 in which the collection of the tissues has been completed. Here, in the confirmation image 102A, the position of the lymph node image 106 where “Done” is displayed corresponds to the position of the lymph node 67 in which the collection has been completed. In the example illustrated in FIG. 8, the display of “Done” is added to the lymph node image 106. In addition, this is only an example. For example, for the lymph node image 106 in which the collection of the tissues has been completed, characters or a symbol other than “Done” may be displayed, or a color or a pattern may be changed. The position of the lymph node 67 in which the collection of the tissues has been completed is an example of a “first position” according to the technology of the present disclosure, and the position of the lymph node image 106 corresponding to the position of the lymph node 67 in which the collection of the tissues has been completed is an example of a “second position” according to the technology of the present disclosure.

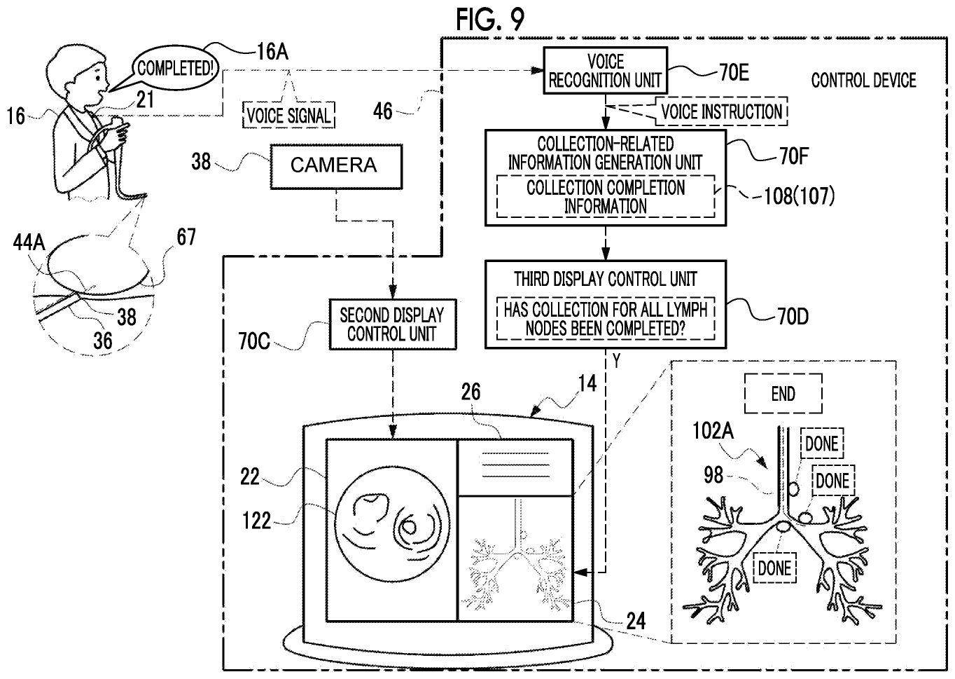

For example, as illustrated in FIG. 9, after the collection of tissues in a certain lymph node 67 is completed, the doctor 16 moves the distal end part 36 along the route 68 indicated by the bronchial pathway 98 and collects tissues in the next lymph node 67. The voice recognition unit 70E recognizes the voice indicated by the voice signal input from the microphone 21. The collection-related information generation unit 70F receives the voice instruction indicating the completion of the biological test output by the voice recognition unit 70E. In addition, the collection-related information generation unit 70F generates the collection completion information 108. The third display control unit 70D updates the confirmation image 102A displayed on the second screen 24 according to the collection completion information 108.

The biological test is repeatedly performed along the route 68, and a voice is input by the doctor 16 whenever the collection of the tissues is completed. Then, the collection-related information generation unit 70F generates the collection completion information 108 according to the voice instruction. In the example illustrated in FIG. 9, the third display control unit 70D determines whether or not the collection of the tissues for all of the lymph nodes 67 has been completed. The third display control unit 70D compares the lymph node 67, in which the collection of the tissues has been completed, indicated by the collection completion information 108 with the lymph node 67 to be subjected to the biopsy indicated by the biopsy route information 99 (see FIG. 7) to determine whether or not the collection of the tissues for all of the lymph nodes 67 has been completed.

In a case in which the third display control unit 70D determines that the collection of the tissues for all of the lymph nodes 67 has been completed, the third display control unit 70D updates the confirmation image 102A displayed on the second screen 24. The third display control unit 70D displays that the biological test has been ended on the second screen 24. In the example illustrated in FIG. 9, “Ended” is displayed on an upper side of the confirmation image 102A.

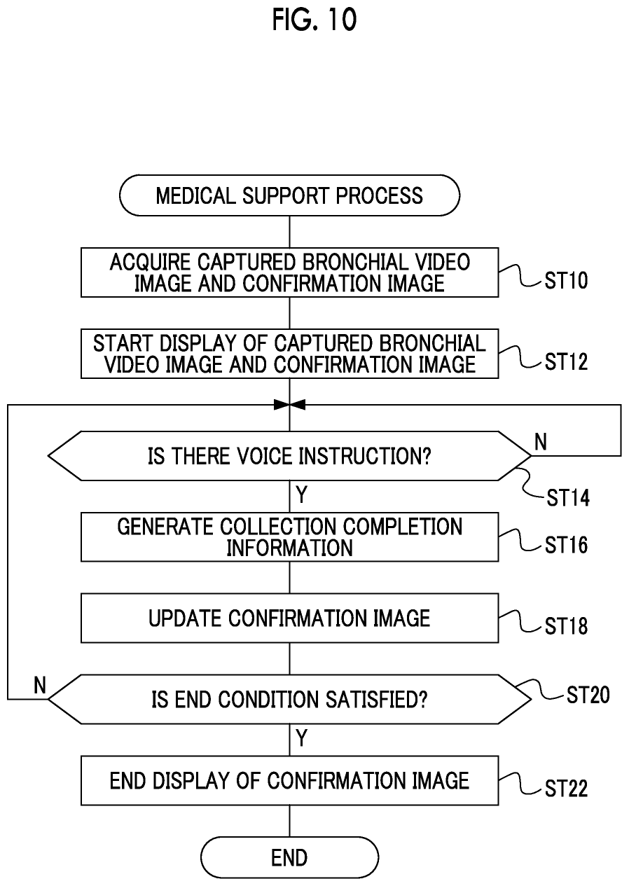

Next, the operation of the endoscope system 10 will be described with reference to FIG. 10. An example of a flow of the medical support process performed by the processor 70 of the control device 46 will be described with reference to FIG. 10.

In the medical support process illustrated in FIG. 10, first, in Step ST10, the second display control unit 70C acquires the captured bronchial video image 122 (see FIG. 7). In addition, the third display control unit 70D acquires the confirmation image 102A from the NVM 92 of the biopsy route generation device 82. After the process in Step ST10 is performed, the medical support process proceeds to Step ST12.

In Step ST12, the second display control unit 70C displays the captured bronchial video image 122 on the first screen 22 of the display device 14. In addition, the third display control unit 70D displays the confirmation image 102A on the second screen 24 of the display device 14. After the process in Step ST12 is performed, the medical support process proceeds to Step ST14.

In Step ST14, the voice recognition unit 70E determines whether or not a voice instruction is given by the doctor 16. In Step ST14, in a case in which the voice instruction is not given by the doctor 16, the determination result is “No”, and the process in Step ST14 is performed again. In a case in which the voice instruction is given by the doctor 16 in Step ST14, the determination result is “Yes”, and the medical support process proceeds to Step ST16.

In Step ST16, the collection-related information generation unit 70F generates the collection completion information 108 according to the voice instruction given in Step ST14. After the process in Step ST16 is performed, the medical support process proceeds to Step ST18.

In Step ST18, the third display control unit 70D updates the confirmation image 102A displayed on the second screen 24 according to the collection completion information 108 generated in Step ST16. For example, the third display control unit 70D adds the display of “Done” to the lymph node image 106. After the process in Step ST18 is performed, the medical support process proceeds to Step ST20.

In Step ST20, the third display control unit 70D determines whether or not a condition for ending the medical support process (hereinafter, referred to as an “end condition”) is satisfied. An example of the end condition is that the collection of the tissues for the lymph node 67 to be subjected to the biopsy has been completed. In a case in which the end condition is not satisfied in Step ST20, the determination result is “No”, and the medical support process proceeds to Step ST14. In a case in which the end condition is satisfied in Step ST20, the determination result is “Yes”, and the medical support process proceeds to Step ST22.

In Step ST22, the third display control unit 70D ends the display of the confirmation image 102A on the second screen 24. After the process in Step ST22 is performed, the medical support process ends.

As described above, in the endoscope system 10 according to this embodiment, in a case in which tissues are collected from a plurality of lymph nodes 67 present in the edge parts of the bronchi 66, the processor 70 of the control device 46 outputs the collection-related information 107, which is information indicating the collection state of the tissues for each of the plurality of lymph nodes 67, to the display device 14. The confirmation image 102A displayed on the second screen 24 of the display device 14 is updated according to the output collection-related information 107. For example, in the confirmation image 102A, the display of “Done” is added to the lymph node image 106 corresponding to the lymph node 67 in which the collection of the tissues has been completed. This makes it possible for the user to ascertain the progress of the collection of the tissues and to check whether or not there is any omission in the collection. Therefore, according to this configuration, the collection state of the tissues is ascertained. For example, the collection state of the tissues is more accurately ascertained, as compared to a case in which information related to the collection of the tissues is collectively output to the display device 14 after the collection of all of the tissues is ended.

Further, in the endoscope system 10 according to this embodiment, the collection-related information 107 includes the collection completion information 108. The collection completion information 108 is information indicating the completion of the collection of the tissues for each of the plurality of lymph nodes 67. In the confirmation image 102A, the display of “Done” is added to the lymph node image 106 corresponding to the lymph node 67, in which the collection of the tissues has been completed, by the output of the collection completion information 108 to the display device 14. This makes it possible for the user to ascertain the completion of the collection of the tissues for each lymph node 67. Therefore, according to this configuration, the collection state of the tissues is ascertained.

In addition, in the endoscope system 10 according to this embodiment, the collection-related information 107 is generated on the basis of the instruction (for example, a voice input of “Completed” by the doctor) received by the receiving device (for example, the microphone 21). Therefore, the collection-related information 107, in which the instruction from the user has been reflected, is output to the display device 14. As a result, according to this configuration, the instruction from the user is accurately reflected in the display of the collection state of the tissues.

Further, in the endoscope system 10 according to this embodiment, in the confirmation image 102A which is an image showing the bronchi 66 and the lymph nodes 67, the collection state of the tissues indicated by the collection-related information 107 for each lymph node 67 is displayed for the lymph node image 106 displayed at the position corresponding to the lymph node 67. This makes it easy to ascertain the correspondence relationship between the collection positions of the tissues in the bronchi 66 and the lymph nodes 67 present around the bronchi 66 and the collection state of the tissues. Therefore, according to this configuration, the collection state of the tissues is accurately displayed.

In addition, in the endoscope system 10 according to this embodiment, the user selects the order in which the tissues are collected from the plurality of lymph nodes 67. That is, the order in which the tissues are collected from the plurality of lymph nodes 67 is predetermined. Therefore, according to this configuration, a processing load required for displaying the collection state of the tissues is reduced.

Further, in the above-described embodiment, the aspect has been described in which the microphone 21 as the receiving device outputs the voice 16A uttered by the doctor 16 as a voice signal to the voice recognition unit 70E and the collection-related information generation unit 70F receives the voice instruction output by the voice recognition unit 70E in a case in which the voice recognition unit 70E recognizes the voice instruction which is an instruction by the voice 16A of the doctor 16. However, the technology of the present disclosure is not limited to this aspect. For example, the doctor 16 may input that the collection of the tissues has been completed through the touch panel 54, the mouse, and/or the keyboard as the receiving device. In addition, other examples of the receiving device include a portable terminal (for example, a tablet terminal), a foot switch, and/or a switch provided in the bronchoscope 18.

Further, in the above-described embodiment, the aspect has been described in which the confirmation image 102A is displayed in order to confirm the collection state of the tissues. However, the technology of the present disclosure is not limited to this aspect. For example, instead of the confirmation image 102A or together with the confirmation image 102A, a table in which the lymph nodes 67 to be collected are displayed, a checklist, and/or a numerical value (for example, percentage indicating the progress) indicating the collection state may be displayed.

Furthermore, in the above-described embodiment, the aspect in which the confirmation image 102A is displayed on the second screen 24 has been described. However, the technology of the present disclosure is not limited to this aspect. For example, the confirmation image 102A may be displayed in a window different from the window in which the captured bronchial video image 122 is displayed. In addition, the confirmation image 102A may be displayed on a display device different from the display device 14 on which the captured bronchial video image 122 is displayed.

Moreover, in the above-described embodiment, the aspect in which the collection-related information 107 is output to the display device 14 has been described. However, the technology of the present disclosure is not limited to this aspect. For example, the collection-related information 107 may be output to a speaker, instead of to the display device 14 or together with the display device 14, such that notification of the collection state of the tissues is sent to the user by voice. In addition, the collection-related information 107 may be stored in the NVM 74. Further, the collection-related information 107 may be output to an external device (for example, a personal computer).

Further, in the above-described embodiment, the aspect in which the display device 14 is a liquid crystal display of the endoscope apparatus 12 has been described. However, the technology of the present disclosure is not limited to this aspect. For example, the display device 14 may be a display (a head-mounted display and/or a screen of a portable terminal (for example, a tablet)) that is separate from the endoscope apparatus 12.

### First Modification Example

In the first embodiment, the aspect in which the collection-related information generation unit 70F generates the collection completion information 108 has been described. However, the technology of the present disclosure is not limited to this aspect. In a first modification example, the collection-related information generation unit 70F generates the collection completion information 108, and the collection completion information 108 is associated with a bronchoscope image 122A.

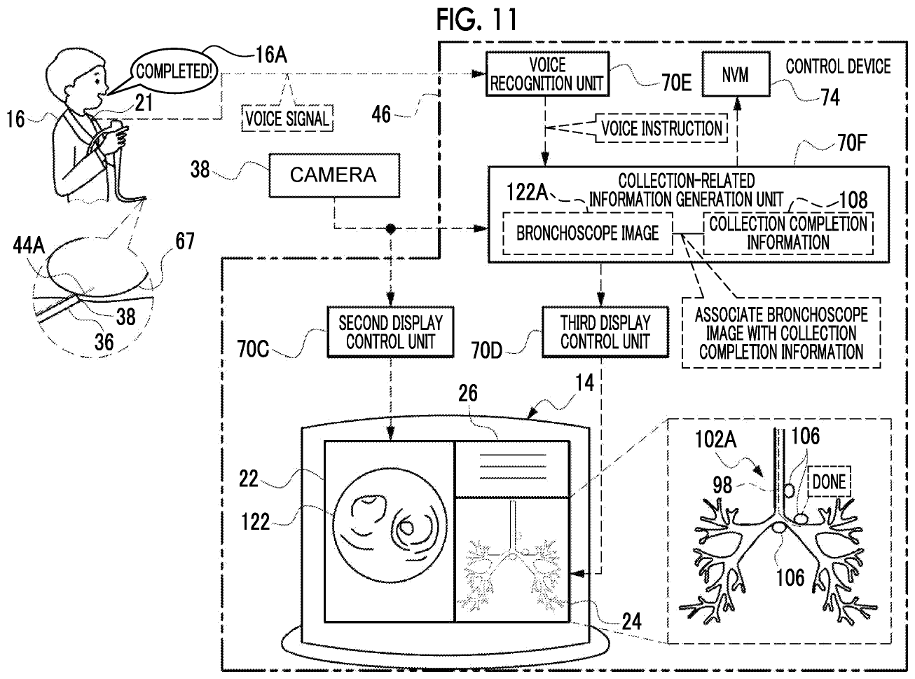

For example, as illustrated in FIG. 11, the collection-related information generation unit 70F receives a voice instruction indicating the completion of the biological test output by the voice recognition unit 70E. The collection-related information generation unit 70F generates the collection completion information 108 according to the voice instruction. In addition, the collection-related information generation unit 70F acquires the captured bronchial video image 122 from the camera 38. The collection-related information generation unit 70F extracts a still image of a predetermined frame from the captured bronchial video image 122 to generate the bronchoscope image 122A. For example, the collection-related information generation unit 70F generates, as the bronchoscope image 122A, a still image of a frame at the time when the voice instruction is received.

The collection-related information generation unit 70F associates the collection completion information 108 with the bronchoscope image 122A. For example, the collection-related information generation unit 70F uses the collection completion information 108 as accessory information of the bronchoscope image 122A and associates the collection completion information 108 with the bronchoscope image 122A. In this case, the collection completion information 108 may include information related to a collection date and time and the lymph node 67 in which the collection has been performed.

The third display control unit 70D updates the confirmation image 102A displayed on the second screen 24 according to the collection completion information 108. In addition, the third display control unit 70D stores the bronchoscope image 122A associated with the collection completion information 108 in the NVM 74.

As described above, in the endoscope system 10 according to the first modification example, the collection completion information 108 is associated with the bronchoscope image 122A obtained for each of the plurality of lymph nodes 67. During or after the collection of the tissues, it may be necessary to ascertain a test image (for example, the bronchoscope image 122A) and the progress of the collection of the tissues in association with each other. Therefore, according to this configuration, the correspondence relationship between the collection state of the tissues and the image showing the collection of the tissues is ascertained.

### Second Modification Example

In the above-described embodiment, the aspect in which the collection-related information generation unit 70F generates the collection completion information 108 as the collection-related information 107 has been described. However, the technology of the present disclosure is not limited to this aspect. In a second modification example, the collection-related information generation unit 70F generates collection number information 110, which is information indicating the number of times the tissues are collected, as the collection-related information 107.

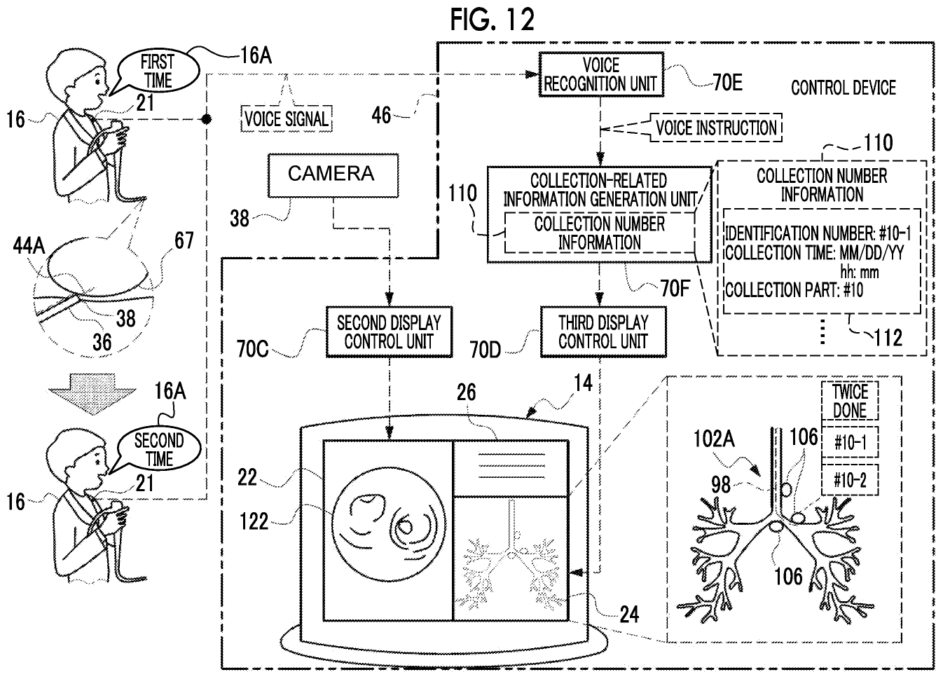

For example, as illustrated in FIG. 12, the microphone 21 outputs the voice 16A uttered by the doctor 16 as a voice signal to the voice recognition unit 70E. In a case in which the voice recognition unit 70E recognizes a voice instruction which is an instruction by the voice 16A of the doctor 16, the collection-related information generation unit 70F receives the voice instruction output by the voice recognition unit 70E. The voice instruction includes an instruction indicating the number of biological tests. For example, a voice instruction “first time” issued by the doctor 16 is an instruction indicating that a first operation of collecting the tissues of the lymph node 67 with the puncture needle 44A has been completed. In addition, here, for convenience of explanation, the voice instruction “first time” is given as an example. However, this is only an example, and any voice instruction may be used as long as it can specify that a first tissue collection operation has been completed.

The collection-related information generation unit 70F generates the collection number information 110 according to the voice instruction. The collection number information 110 is information indicating the number of times the collection of the tissues is performed for one lymph node 67 among a plurality of lymph nodes 67 to be collected. The collection number information 110 is an example of “collection number information” according to the technology of the present disclosure.

The collection number information 110 includes collection identification information 112 for identifying collection. For example, the collection identification information 112 includes information indicating an identification number for identifying each of a plurality of collections in a case in which the collection of the tissues is performed for a certain lymph node 67 a plurality of times. In the example illustrated in FIG. 12, “#10-1” is given as the identification number of the first tissue collection operation. The collection identification information 112 is an example of “collection identification information” according to the technology of the present disclosure.

In addition, the collection identification information 112 may include information indicating a collection time, which is the time when the tissues were collected, and information indicating a collection part (for example, which lymph node 67 the collection was performed for), which is a part in which the tissues were collected. The third display control unit 70D updates the confirmation image 102A displayed on the second screen 24 according to the collection number information 110.

After performing the first tissue collection operation, the doctor 16 performs a second tissue collection operation for the same lymph node 67. The microphone 21 outputs a voice (for example, a voice “second time”) uttered by the doctor 16 as a voice signal to the voice recognition unit 70E. The voice recognition unit 70E recognizes the voice indicated by the voice signal input from the microphone 21. The collection-related information generation unit 70F receives a voice instruction indicating the completion of the second tissue collection output by the voice recognition unit 70E. The collection-related information generation unit 70F updates the collection number information 110 according to the voice instruction.

The third display control unit 70D updates the confirmation image 102A displayed on the second screen 24 according to the collection number information 110. The third display control unit 70D adds display indicating that the collection of the tissues has been completed to the lymph node image 106 corresponding to the lymph node 67 in which the collection of the tissues has been completed. In the example illustrated in FIG. 12, the display of “Twice done”, “#10-1”, and “#10-2” is added to the lymph node image 106. In addition, this is only an example. For example, for the lymph node image 106 corresponding to the lymph node 67 in which the collection of the tissues has been completed, characters other than “Twice done”, “#10-1”, and “#10-2” may be displayed, or a color or a pattern may be changed.

As described above, in the endoscope system 10 according to the second modification example, the collection-related information 107 includes the collection number information 110 which is information indicating the number of times the collection of the tissues is performed for each lymph node 67. The display of the number of collections is added to the lymph node image 106 corresponding to the lymph node 67 in the confirmation image 102A by the output of the collection number information 110 to the display device 14. This makes it possible for the user to ascertain the number of times the collection of the tissues is performed for each lymph node 67. Therefore, according to this configuration, the collection state of the tissues is ascertained.

In addition, in the endoscope system 10 according to the second modification example, the collection-related information 107 includes the collection identification information 112 which is information for identifying each of a plurality of tissue collections in a case in which the collection of the tissues in the lymph node 67 is performed a plurality of times. The output of the collection identification information 112 makes it possible to identify each collection even in a case in which a plurality of collections are performed for each lymph node 67. Therefore, according to this configuration, the collection state of the tissues is ascertained.

Further, in the second modification example, the aspect in which the collection number information 110 is included as the collection-related information 107 has been described. However, the technology of the present disclosure is not limited to this aspect. The collection-related information 107 may include the collection completion information 108 and the collection number information 110. The collection completion information 108 and the collection number information 110 as the collection-related information 107 are output to the display device 14. Therefore, in the confirmation image 102A, the display of “Done” is added to the lymph node image 106 corresponding to the lymph node 67 in which the collection of the tissues has been completed. In addition, in the confirmation image 102A, the display of the number of collections is added to the lymph node image 106 corresponding to the lymph node 67. This makes it possible for the user to ascertain the completion of the collection of the tissues and the number of tissue collections for each lymph node 67. Therefore, according to this configuration, the collection state of the tissues is ascertained.

### Third Modification Example

In the above-described second modification example, the aspect in which the collection of the tissues is performed for a certain lymph node 67 a plurality of times has been described. However, the technology of the present disclosure is not limited to this aspect. In a third modification example, in a case in which the collection of the tissues is performed a plurality of times, association is made for the result of a pathological test for the tissues collected in the biological test.

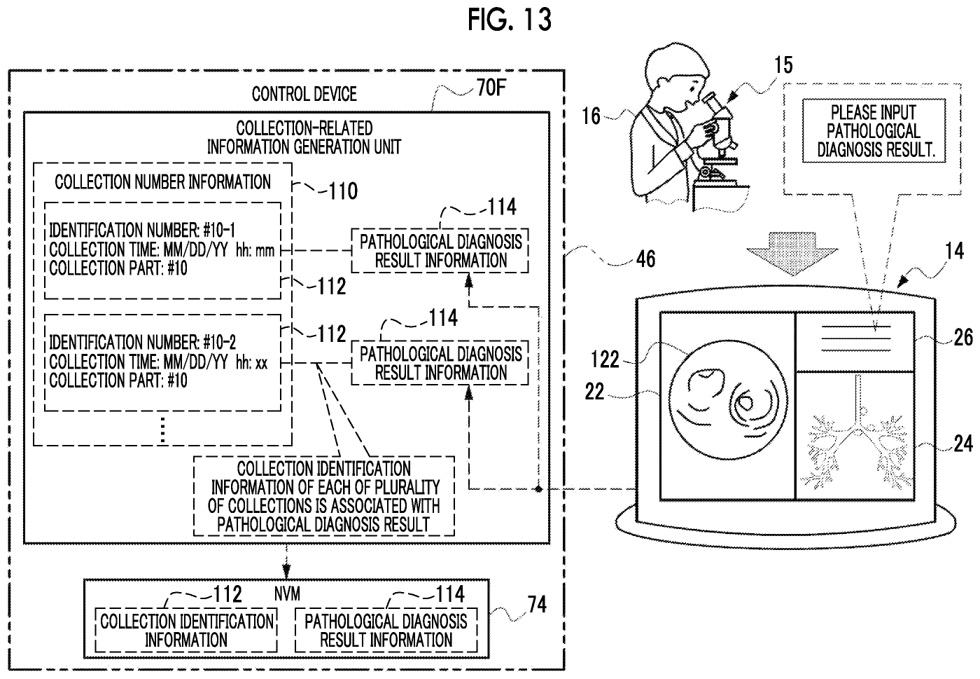

For example, as illustrated in FIG. 13, the doctor 16 performs the pathological test for the tissues collected in the biological test. For example, the doctor 16 observes the tissues using a microscope 15 to perform the pathological test. The result of the pathological test is received through the receiving device (for example, the touch panel 54). In the example illustrated in FIG. 13, information for prompting the input of the result of the pathological test is displayed on the third screen 26.

In the control device 46, the collection-related information generation unit 70F acquires pathological diagnosis result information 114 which is information indicating the result of the pathological test received through the receiving device. The collection-related information generation unit 70F associates the pathological diagnosis result information 114 with the collection identification information 112. The collection-related information generation unit 70F associates the collection identification information 112 indicating each of a plurality of collections with the pathological diagnosis result information 114 indicating the result of a pathological diagnosis for each of the tissues obtained by the plurality of collections. The collection-related information generation unit 70F stores the collection identification information 112 and the pathological diagnosis result information 114 in the NVM 74.

As described above, in the endoscope system 10 according to the third modification example, the collection identification information 112 is associated with the pathological diagnosis result information 114 which is information indicating the result of the pathological diagnosis obtained by the collection of the tissues for each of a plurality of tissue collections in the lymph node 67. In some cases, it is necessary to ascertain the result of the pathological diagnosis and the progress of the collection of the tissues in association with each other during or after the collection of the tissues. Therefore, according to this configuration, the correspondence relationship between the collection state of the tissues and the result of the pathological diagnosis is ascertained.

### Second Embodiment

In the first embodiment, the aspect has been described in which the completion of the collection of the tissues is received by the voice instruction from the doctor 16. However, the technology of the present disclosure is not limited to this aspect. In a second embodiment, the collection completion information 108 indicating the completion of the collection of the tissues is generated on the basis of the captured bronchial video image 122.

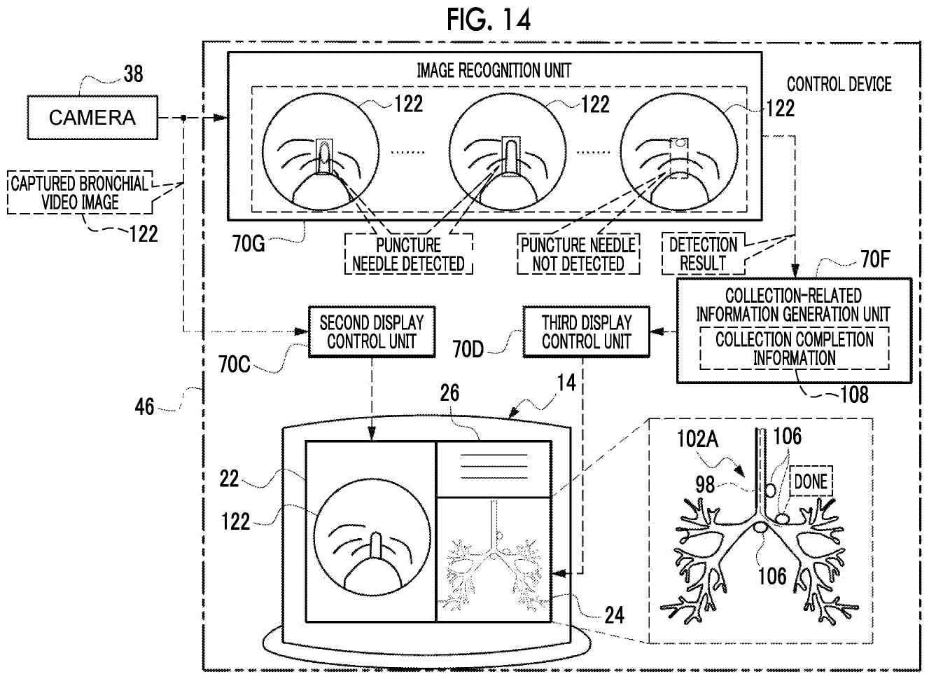

For example, as illustrated in FIG. 14, a processor 70 according to the second embodiment comprises an image recognition unit 70G. The image recognition unit 70G acquires the captured bronchial video image 122 from the camera 38. The captured bronchial video image 122 is an example of a “medical image”, a “real-time image”, and an “optical image” according to the technology of the present disclosure. The captured bronchial video image 122 is, for example, a live view image. The image recognition unit 70G performs an image recognition process on the captured bronchial video image 122 to detect an image region indicating the puncture needle 44A. The image region is detected, for example, by an image recognition process based on an AI method. For example, the image recognition unit 70G detects the puncture needle 44A using a trained model for detecting a puncture needle which is stored in the NVM 74 (see FIG. 4) in advance. Further, in a case in which the detection state of the puncture needle 44A continues for a predetermined time (that is, a time that takes into account agitation which is the forward and backward movement of the puncture needle 44A that may be performed during the collection of the tissues), the image recognition unit 70G may determine that the puncture needle 44A has been detected. In addition, the image recognition process based on the AI method has been described here as an example. However, this is only an example. For example, the puncture needle 44A may be detected by an image recognition process based on a template matching method. The image recognition unit 70G outputs a detection result in a case in which the puncture needle 44A is not detected after the detection of the puncture needle 44A. The case in which the puncture needle 44A is not detected means, for example, a case in which the image region indicating the puncture needle 44A is not included in several frames (for example, 3 frames) of the captured bronchial video image 122. The collection-related information generation unit 70F generates the collection completion information 108 on the basis of the detection result output from the image recognition unit 70G. The third display control unit 70D updates the confirmation image 102A displayed on the second screen 24 according to the collection completion information 108. In addition, the example in which the collection completion information 108 is generated in a case in which the completion of the collection of the tissues is detected has been described here. However, this is only an example. For example, in a case in which the collection of the tissues is detected (for example, in a case in which the insertion of the puncture needle 44A into the bronchus 66 is detected), the collection completion information 108 may be generated.

As described above, in the endoscope system 10 according to the second embodiment, the collection completion information 108 is generated on the basis of the captured bronchial video image 122 indicating the collection of the tissues in the lymph node 67. Therefore, the collection-related information 107 reflects the collection state of the tissues indicated by the captured bronchial video image 122. Therefore, according to this configuration, the collection state of the tissues is ascertained.

Further, in the endoscope system 10 according to the second embodiment, the captured bronchial video image 122 is a real-time image. The collection-related information 107 is obtained from a real-time image showing an aspect of the collection of the tissues. Therefore, the collection completion information 108 reflects the current collection state of the tissues indicated by the real-time image. As a result, according to this configuration, the collection state of the tissues is ascertained.

Further, in the endoscope system 10 according to the second embodiment, the captured bronchial video image 122 is an optical image. The collection completion information 108 is obtained by an optical image showing an aspect of the collection of the tissues. Therefore, the collection completion information 108 reflects the current collection state of the tissues indicated by the optical image. As a result, according to this configuration, the collection state of the tissues is ascertained.

In addition, in the endoscope system 10 according to the second embodiment, the collection completion information 108 is generated in a case in which the tissues are collected in the lymph node 67 in the captured bronchial video image 122. Therefore, according to this configuration, the collection state of the tissues is ascertained.

### Fourth Modification Example

In the second embodiment, the aspect in which the collection completion information 108 is generated on the basis of the captured bronchial video image 122 has been described. However, the technology of the present disclosure is not limited to this aspect. In a fourth modification example, the collection number information 110 is generated on the basis of the captured bronchial video image 122.

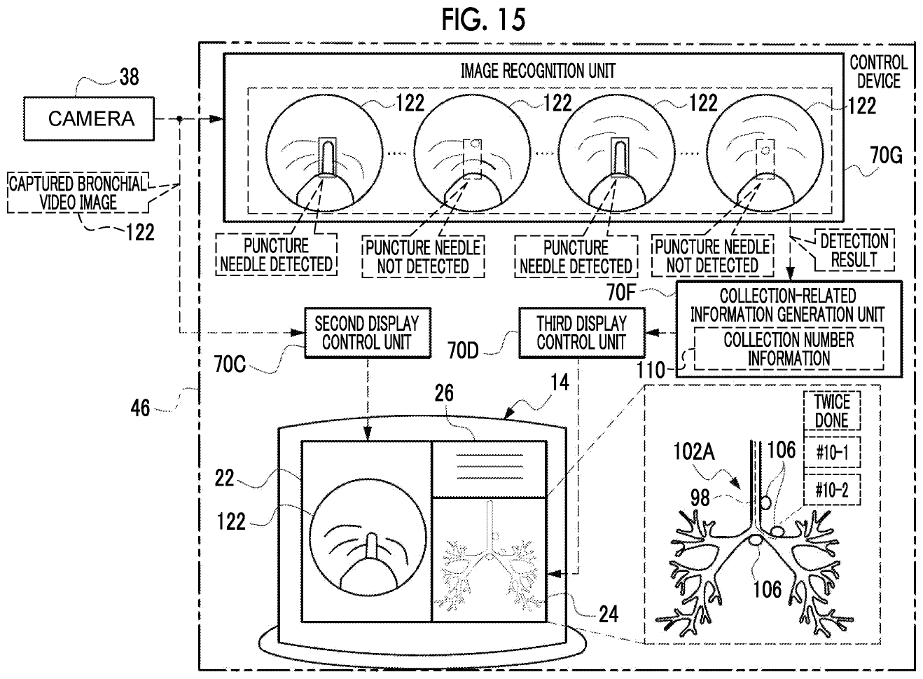

For example, as illustrated in FIG. 15, the image recognition unit 70G acquires the captured bronchial video image 122 from the camera 38. The image recognition unit 70G performs image processing on the captured bronchial video image 122 to detect the image region indicating the puncture needle 44A. The image recognition unit 70G outputs a detection result in a case in which the puncture needle 44A is not detected after the detection of the puncture needle 44A. The case in which the puncture needle 44A is not detected means, for example, a case in which the image region indicating the puncture needle 44A is not included in several frames (for example, 3 frames) of the captured bronchial video image 122.

The collection-related information generation unit 70F generates the collection number information 110 on the basis of the detection result output from the image recognition unit 70G. The collection-related information generation unit 70F generates the collection number information 110, using the number of times the puncture needle 44A is detected, which is indicated by the detection result, as the number of times the tissues are collected. In an example illustrated in FIG. 15, the image recognition unit 70G detects the puncture needle 44A twice, and the collection-related information generation unit 70F generates the collection number information 110 indicating that the tissues have been collected twice. The third display control unit 70D updates the confirmation image 102A displayed on the second screen 24 according to the collection number information 110.

As described above, in the endoscope system 10 according to the fourth modification example, the collection number information 110 is generated on the basis of the captured bronchial video image 122 showing the collection of the tissues in the lymph node 67. Therefore, the collection number information 110 reflects the collection state of the tissues indicated by the captured bronchial video image 122. As a result, according to this configuration, the collection state of the tissues is ascertained.

### Third Embodiment

In the second embodiment, the aspect in which the collection completion information 108 is generated on the basis of the captured bronchial video image 122 has been described. However, the technology of the present disclosure is not limited to this aspect. In a third embodiment, the completion of the collection of the tissues is received by a voice instruction from the doctor 16 with respect to the result generated on the basis of the captured bronchial video image 122.

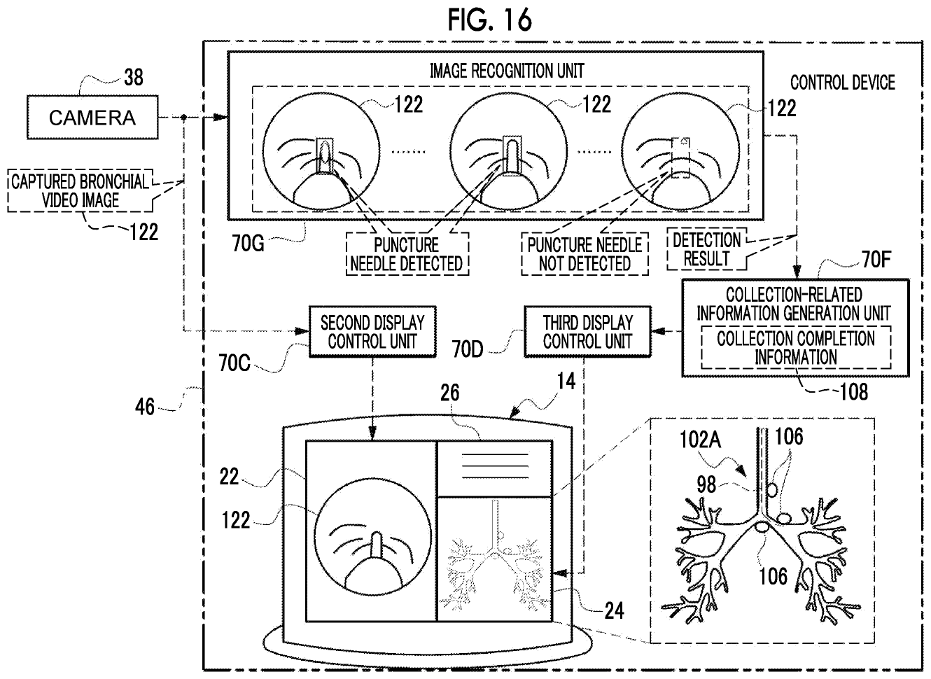

For example, as illustrated in FIG. 16, the image recognition unit 70G acquires the captured bronchial video image 122 from the camera 38. The image recognition unit 70G performs image processing on the captured bronchial video image 122 to detect the image region indicating the puncture needle 44A. The image recognition unit 70G outputs a detection result in a case in which the puncture needle 44A is not detected after the detection of the puncture needle 44A. The collection-related information generation unit 70F generates the collection completion information 108 on the basis of the detection result output from the image recognition unit 70G and outputs the collection completion information 108 to the third display control unit 70D.

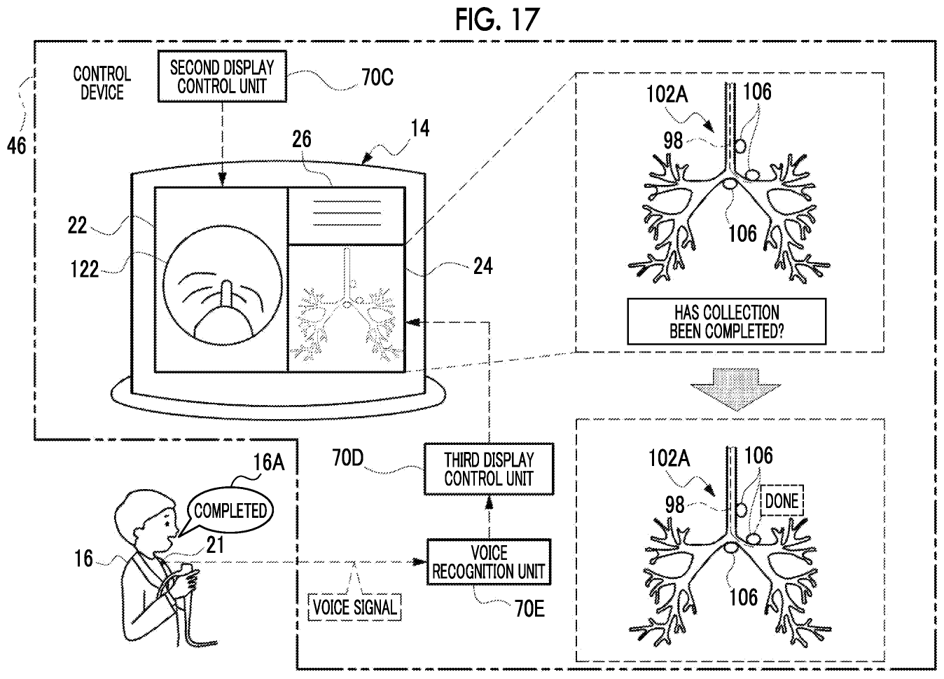

For example, as illustrated in FIG. 17, the third display control unit 70D performs display for allowing the user to confirm that the collection of the tissues has been completed on the second screen 24. In the example illustrated in FIG. 17, a text “Has the collection been completed?” is displayed in a lower portion of the confirmation image 102A on the second screen 24.

The microphone 21 outputs a voice 16A (for example, a voice “Completed”) uttered by the doctor 16 as a voice signal to the voice recognition unit 70E. In a case in which the voice recognition unit 70E recognizes a voice instruction which is an instruction by the voice of the doctor 16, the third display control unit 70D receives the voice instruction output by the voice recognition unit 70E. The third display control unit 70D updates the confirmation image 102A displayed on the second screen 24 according to the collection completion information 108. The third display control unit 70D adds display indicating that the collection of the tissues has been completed to the lymph node image 106 corresponding to the lymph node 67 in which the collection of the tissues has been completed.

As described above, in the endoscope system 10 according to the third embodiment, the receiving device (for example, the microphone 21) receives the voice instruction from the doctor 16. The instruction received by the receiving device is an instruction with respect to the result generated on the basis of the captured bronchial video image 122 indicating the collection of the tissues in the lymph node 67. Therefore, it is possible to get the confirmation on the state of the biological test based on the captured bronchial video image 122 from the doctor 16. As a result, according to this configuration, the collection state of the tissues is ascertained.

In the third embodiment, the aspect in which whether or not the collection of the tissues has been completed is displayed (for example, the text “Has the collection been completed?” is displayed) has been described. However, the technology of the present disclosure is not limited to this aspect. For example, instead of the display of whether or not the collection has been completed or together with the display of whether or not the collection has been completed, display for confirming the number of tissue collections may be performed.

In addition, in the third embodiment, the aspect in which whether or not the collection of the tissues has been completed is displayed has been described. However, the technology of the present disclosure is not limited to this aspect. For example, after the image recognition unit 70G detects that the collection of the tissues has been completed on the basis of the captured bronchial video image 122, a text for receiving correction may be displayed on the second screen 24 (for example, a text “Do you want to correct it?” may be displayed). The receiving device (for example, the microphone 21) receives a correction instruction from the user. In this case, in a case in which the correction is input by the user, the image recognition unit 70G performs image processing for detecting the collection of the tissues in the captured bronchial video image 122 again. In a case in which the correction is not input by the user, the image recognition unit 70G confirms that the collection of the tissues has been completed. Therefore, it is possible to get the confirmation on the state of the biological test based on the captured bronchial video image 122 from the doctor 16. As a result, according to this configuration, the collection state of the tissues is ascertained.

### Fifth Modification Example

In each of the above-described embodiments, the aspect in which an optical image is used as the captured bronchial video image 122 has been described. However, the technology of the present disclosure is not limited to this aspect. In a fifth modification example, a bronchial ultrasound video image 126 is used instead of the captured bronchial video image 122.

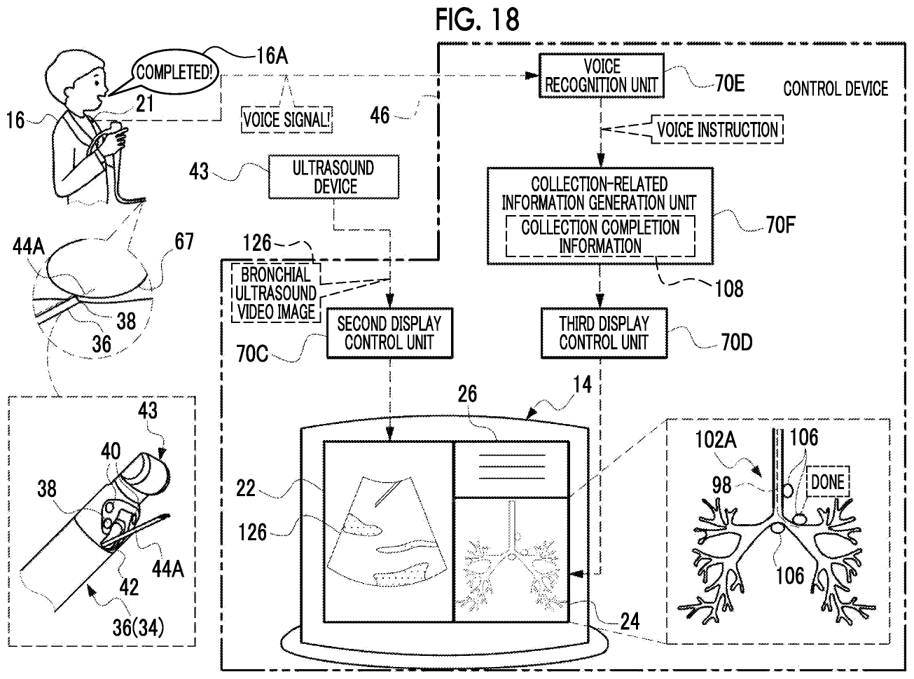

For example, as illustrated in FIG. 18, the camera 38, the illumination device 40, the treatment tool opening 42, and an ultrasound device 43 are provided in the distal end part 36 of the insertion portion 34. The ultrasound device 43 emits ultrasonic waves from an ultrasound transducer to perform ultrasonography on the inside of the bronchus. In the control device 46, the second display control unit 70C acquires the bronchial ultrasound video image 126 from the ultrasound device 43. The bronchial ultrasound video image 126 is an example of the endoscope image 28 illustrated in FIG. 1. The bronchial ultrasound video image 126 is an example of a “medical image”, an “ultrasound image”, and a “real-time image” according to the technology of the present disclosure. The bronchial ultrasound video image 126 is a video image obtained by performing ultrasonography on the inside of the trachea 64 and the inside of the bronchus 66 (see FIG. 3) along the route 68 (see FIG. 3) with the ultrasound device 43. The second display control unit 70C displays the bronchial ultrasound video image 126 on the first screen 22 of the display device 14.

The microphone 21 outputs the voice 16A uttered by the doctor 16 as a voice signal to the voice recognition unit 70E. In a case in which the voice recognition unit 70E recognizes a voice instruction which is an instruction by the voice 16A of the doctor 16, the collection-related information generation unit 70F receives the voice instruction output by the voice recognition unit 70E. The collection-related information generation unit 70F generates the collection completion information 108 according to the voice instruction. The third display control unit 70D updates the confirmation image 102A displayed on the second screen 24 according to the collection completion information 108.

As described above, in the endoscope system 10 according to the fifth modification example, the bronchial ultrasound video image 126 is an ultrasound image. The ultrasound image is an image obtained by performing ultrasonography on the bronchus 66 and the lymph nodes 67 around the bronchus 66. The use of the ultrasound image makes it possible to obtain information of, for example, the positional relationship between the lymph node 67 and the puncture needle 44A which is not capable of being ascertained only by the optical image. As a result, according to this configuration, the collection state of the tissues is ascertained.

### Sixth Modification Example

In the fifth modification example, the aspect in which the completion of the collection of the tissues is received by the voice instruction from the doctor 16 has been described. However, the technology of the present disclosure is not limited to this aspect. In a sixth modification example, the collection completion information 108 indicating the completion of the collection of the tissues may be generated on the basis of the bronchial ultrasound video image 126.

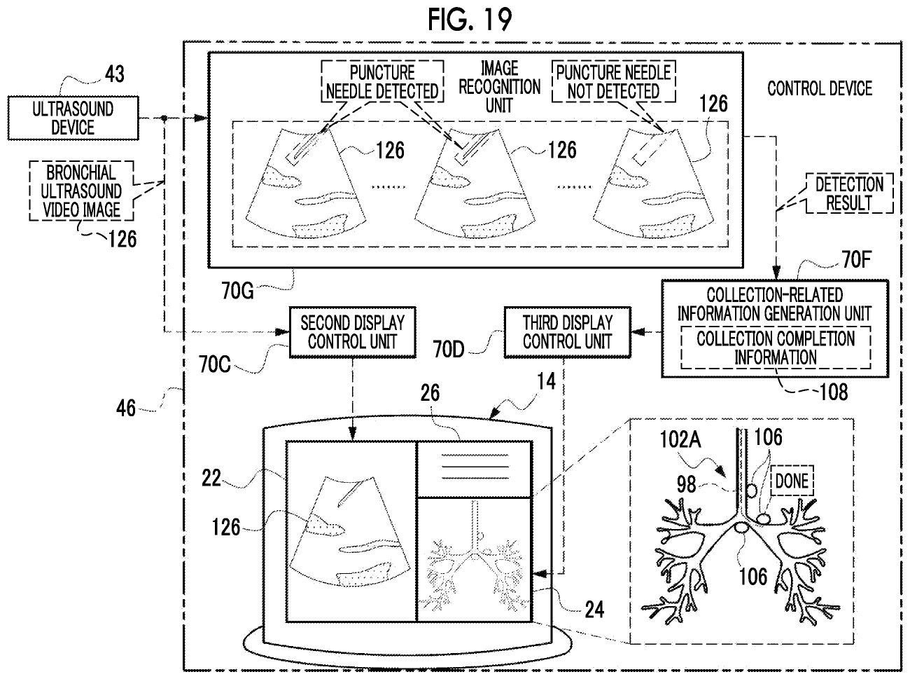

For example, as illustrated in FIG. 19, the image recognition unit 70G acquires the bronchial ultrasound video image 126 from the ultrasound device 43. The image recognition unit 70G performs image processing on the bronchial ultrasound video image 126 to detect the image region indicating the puncture needle 44A. The image recognition unit 70G outputs a detection result in a case in which the puncture needle 44A is not detected after the detection of the puncture needle 44A. The collection-related information generation unit 70F generates the collection completion information 108 on the basis of the detection result output from the image recognition unit 70G. The third display control unit 70D updates the confirmation image 102A displayed on the second screen 24 according to the collection completion information 108.

As described above, in the endoscope system 10 according to the sixth modification example, the collection completion information 108 is generated on the basis of the bronchial ultrasound video image 126 indicating the collection of the tissues in the lymph node 67. Therefore, the collection completion information 108 reflects the collection state of the tissues indicated by the bronchial ultrasound video image 126. As a result, according to this configuration, the collection state of the tissues is ascertained.

Further, in the endoscope system 10 according to the sixth modification example, the bronchial ultrasound video image 126 is a real-time image. The collection completion information 108 is obtained by the real-time image showing an aspect of the collection of the tissues. Therefore, the collection completion information 108 reflects the current collection state of the tissues indicated by the real-time image. As a result, according to this configuration, the collection state of the tissues is ascertained.

Further, in the third embodiment, the aspect in which the collection-related information generation unit 70F generates the collection completion information 108 has been described. However, the technology of the present disclosure is not limited to this aspect. Even in a case in which the bronchial ultrasound video image 126 is used, the collection-related information generation unit 70F may generate the collection number information 110 instead of the collection completion information 108 or together with the collection completion information 108, as in the case of the captured bronchial video image 122.

In addition, in each of the above-described embodiments, the aspect in which either the captured bronchial video image 122 or the bronchial ultrasound video image 126 is used has been described. However, the technology of the present disclosure is not limited to this aspect. For example, both the captured bronchial video image 122 and the bronchial ultrasound video image 126 may be used. Further, a roentgen image obtained by the endoscope apparatus 12 may be used instead of or together with the captured bronchial video image 122 and the bronchial ultrasound video image 126.

In each of the above-described embodiments, the bronchoscope 18 is given as an example. However, the technology of the present disclosure is not limited thereto. The technology of the present disclosure is applicable even to endoscopes used to observe a cavity region (for example, a region from an esophagus to a duodenum or a region from an anus to a small intestine) in the body, such as upper gastrointestinal endoscopes or lower gastrointestinal endoscopes. In this case, the cavity region in the body corresponds to the trachea 64 and the bronchus 66 to which the route 68 described in the above-described embodiment has been given.

In each of the above-described embodiments, the aspect in which the first screen 22, the second screen 24, and the third screen 26 are displayed on the display device 14 has been described. However, the first screen 22, the second screen 24, and the third screen 26 may be dispersively displayed by different display devices. In addition, the size of the first screen 22, the size of the second screen 24, and the size of the third screen 26 may be selectively changed.

In each of the above-described embodiments, the aspect in which the medical support process is performed by the processor 70 of the endoscope apparatus 12 has been described. However, the technology of the present disclosure is not limited to this aspect. For example, the device that performs the medical support process may be provided outside the endoscope apparatus 12. An example of the device provided outside the endoscope apparatus 12 is a server. For example, the server is implemented by cloud computing. Here, cloud computing is given as an example. However, this is only an example. For example, the server may be implemented by a mainframe or may be implemented by network computing such as fog computing, edge computing, or grid computing. Here, the server is given as an example of the device provided outside the endoscope apparatus 12. However, this is only an example. For example, at least one personal computer may be used instead of the server. In addition, the medical support process may be dispersively performed by a plurality of devices including the endoscope apparatus 12 and the device provided outside the endoscope apparatus 12.

Further, in each of the above-described embodiments, the aspect in which the medical support processing program 84 is stored in the NVM 74 has been described. However, the technology of the present disclosure is not limited to this aspect. For example, the medical support processing program 84 may be stored in a portable storage medium such as an SSD or a USB memory. The storage medium is a non-transitory computer-readable storage medium. The medical support processing program 84 stored in the storage medium is installed in the computer 69 of the control device 46. The processor 70 performs the medical support process according to the medical support processing program 84.

In each of the above-described embodiments, the computer 69 is given as an example. However, the technology of the present disclosure is not limited thereto, and a device including an ASIC, an FPGA, and/or a PLD may be applied instead of the computer 69. In addition, a combination of a hardware configuration and a software configuration may be used instead of the computer 69.

The following various processors can be used as hardware resources for performing various processes described in each of the above-described embodiments. An example of the processor is a CPU which is a general-purpose processor that executes software, that is, a program, to function as the hardware resource performing the medical support process. In addition, an example of the processor is a dedicated electronic circuit which is a processor having a dedicated circuit configuration designed to perform a specific process, such as an FPGA, a PLD, or an ASIC. Any processor has a memory built in or connected to it, and any processor uses the memory to perform the medical support process.

The hardware resource for performing the medical support process may be configured by one of the various processors or by a combination of two or more processors of the same type or different types (for example, a combination of a plurality of FPGAs or a combination of a processor and an FPGA). Further, the hardware resource for performing the medical support process may be one processor.

A first example of the configuration in which the hardware resource is configured by one processor is an aspect in which one processor is configured by a combination of one or more processors and software and functions as the hardware resource for performing the medical support process. A second example of the configuration is an aspect in which a processor that implements the functions of the entire system including a plurality of hardware resources for performing the medical support process using one IC chip is used. A representative example of this aspect is an SoC. As described above, the medical support process is achieved using one or more of the various processors as the hardware resource.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The content described and illustrated above is a detailed description of portions related to the technology of the present disclosure and is only an example of the technology of the present disclosure. For example, the description of the configurations, functions, operations, and effects is the description of examples of the configurations, functions, operations, and effects of the portions related to the technology of the present disclosure. Therefore, it goes without saying that unnecessary portions may be deleted or new elements may be added or replaced in the content described and illustrated above, without departing from the gist of the technology of the present disclosure. In addition, the description of, for example, common technical knowledge that does not need to be particularly described to enable the implementation of the technology of the present disclosure is omitted in the content described and illustrated above in order to avoid confusion and to facilitate the understanding of the portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case in which the connection of three or more matters is expressed by “and/or”.

All of the documents, the patent applications, and the technical standards described in the specification are incorporated by reference herein to the same extent as each individual document, each patent application, and each technical standard is specifically and individually stated to be incorporated by reference.

What is claimed is:

1. A medical support device comprising:
a processor,
wherein, in a case in which a tissue is collected at a plurality of first positions in an organ, the processor is configured to output collection-related information which is information related to a collection state of the tissue at each of the first positions.

2. The medical support device according to claim 1,
wherein the collection-related information includes collection completion information which is information indicating completion of the collection of the tissue at each of the first positions.

3. The medical support device according to claim 1,
wherein the collection-related information includes collection number information which is information indicating the number of times the tissue is collected at each of the first positions.

4. The medical support device according to claim 1,
wherein the collection-related information includes collection completion information, which is information indicating completion of the collection of the tissue at each of the first positions, and collection number information, which is information indicating the number of times the tissue is collected at each of the first positions.

5. The medical support device according to claim 1,
wherein the collection-related information includes collection identification information which is information for identifying each of a plurality of collections of the tissue in a case in which the collection of the tissue at the first position is performed a plurality of times.

6. The medical support device according to claim 5,
wherein the collection identification information is associated with a pathological diagnosis result obtained by the collection of the tissue for each of the plurality of collections of the tissue at the first position.

7. The medical support device according to claim 1,
wherein the collection-related information is generated on the basis of a medical image showing the collection of the tissue at the first position.

8. The medical support device according to claim 7,
wherein the medical image is a real-time image.

9. The medical support device according to claim 7,
wherein the medical image is an optical image and/or an ultrasound image.

10. The medical support device according to claim 7,
wherein the collection-related information is generated in a case in which the collection of the tissue at the first position is performed in the medical image.

11. The medical support device according to claim 7,
wherein the collection-related information is associated with the medical image obtained for each of the plurality of first positions.

12. The medical support device according to claim 1,
wherein the collection-related information is generated on the basis of an instruction received by a receiving device.

13. The medical support device according to claim 12,
wherein the instruction received by the receiving device is an instruction with respect to a result generated on the basis of a medical image showing the collection of the tissue at the first position.

14. The medical support device according to claim 12,
wherein the instruction received by the receiving device is an instruction received in a case in which the collection of the tissue is detected on the basis of a medical image showing the collection of the tissue at the first position.

15. The medical support device according to claim 1,
wherein the collection state of the tissue indicated by the collection-related information corresponding to the first position is displayed at each second position which is a position corresponding to the first position in an organ image which is an image showing the organ.

16. The medical support device according to claim 15,
wherein the number of times the tissue is collected which is indicated by the collection-related information is displayed at the second position on a display device.

17. The medical support device according to claim 1,
wherein an order in which the tissue is collected at the plurality of first positions is predetermined.

18. A medical support method comprising:
outputting, in a case in which a tissue is collected at a plurality of first positions in an organ, collection-related information which is information related to a collection state of the tissue at each of the first positions.

19. The medical support method according to claim 18, further comprising:
determining whether or not the tissue has been collected on the basis of a medical image showing the collection of the tissue;
receiving a user's instruction with respect to a determination result; and
outputting collection number information indicating the number of times the tissue is collected at each of the first positions according to the instruction.

20. A non-transitory storage medium storing a program that causes a computer to execute a process comprising:
outputting, in a case in which a tissue is collected at a plurality of first positions in an organ, collection-related information which is information related to a collection state of the tissue at each of the first positions.
